SEQUENCE LISTING (21 sequences)

SEQ ID NO: 1 - DNA, 7794 bp, Artificial Sequence
  nucleotide sequence of vector pRN1120-RFP-gRNA(A)
tcatgtttgacagcttatcatcgataatccggagctagcatgcggccgcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttgttccagacacgacgtcagttttagagctagaaatagcaagttaaaataaggctagtccgttatcaacttgaaaaagtggcaccgagtcggtggtgctttttttgttttttatgtctcccgatatcaagcttatcgacgctttccggcatcttccagaccacagtatatccatccgcctcctgttgaggaccggtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaagaattcaaaatggtttcaaaaggtgaagaagataatatggctattattaaagaatttatgagatttaaagttcatatggaaggttcagttaatggtcatgaatttgaaattgaaggtgaaggtgaaggtagaccatatgaaggtactcaaactgctaaattgaaagttactaaaggtggtccattaccatttgcttgggatattttgtcaccacaatttatgtatggttcaaaagcttatgttaaacatccagctgatattccagattatttaaaattgtcatttccagaaggttttaaatgggaaagagttatgaattttgaagatggtggtgttgttactgttactcaagattcatcattacaagatggtgaatttatttataaagttaaattgagaggtactaattttccatcagatggtccagttatgcaaaaaaaaactatgggttgggaagcttcatcagaaagaatgtatccagaagatggtgctttaaaaggtgaaattaaacaaagattgaaattaaaagatggtggtcattatgatgctgaagttaaaactacttataaagctaaaaaaccagttcaattaccaggtgcttataatgttaatattaaattggatattacttcacataatgaagattatactattgttgaacaatatgaaagagctgaaggtagacattcaactggtggtatggatgaattatataaataatctagacaaatcgctcttaaatatatacctaaagaacattaaagctatattataagcaaagatacgtaaattttgcttatattattatacacatatcatatttctatatttttaagatttggttatataatgtacgtaatgcaaaggaaataaattttatacattattgaacagcgtccaagtaactacattatgtgcactaatagtttagcgtcgtgaagactttattgtgtcgcgaaaagtaaaaattttaaaaattagagcaccttgaacttgcgaaaaaggttctcatcaactgtttaaaagatctgagctcgcagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattc

SEQ ID NO: 2 - DNA, 1387 bp, Artificial Sequence
  nucleotide sequence of synthetic expression cassette cFS0017 (pGAL10-GIN11(M86))
ctgaacccgcggcatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgaaaattcaaggaatttcgacggatcaataacagtgtttgtggagcattttctgaatacaataaacccaaaacagaaacttcccttttgtatcactgttctggaaaaggggtgggcggtaataaagctaatagggtgtgtccataagtaatactgaacttggaaatgtgcggctttgcagcattttgtctttctataaaaatgtgtcgttcctttttttcattttttggcgcgtcgcctcggggtcgtatagaatatgcgtcacttttaaaaataagattgcagatcagggcaaaacaagtagcaaatcatagcaagagaccctgatttttgtgacataaatatttttacttctgtgttaggttaactttttatgtaactgtaaatggaatagagttgaggggatagtgcccacaagtcaatatgtttattttgtaaagttgaaagataattatttttatgctcaggtgattttggtgttgaattttctgtaatattaacataagagtaatacattgagtggttagtatatggtgtaaaagtggtataacgcatgtattaagagcagttatacaatatttggggccgctgaatgagatatagatattaaaatgtggataatcatgggctttatgggtaaatggaacagggtatagaccactgaggcaagtgccgtgcataatgatatgagtgcatctagtggcgaacgtggcgagaaaggaagggaagaaagcgagtgccatctgtgcagacaaacgcatcaggatactagtccttga

SEQ ID NO: 3 - DNA, 1385 bp, Artificial Sequence
  nucleotide sequence of synthetic expression cassette cFS0018 (pCUP1-GIN11(M86))
aaccgcggggtacctaaggagatttcagattttttaatggaaagagaagttgtccaaaggagtataattattgacaaggatttggaatctgataatctgggtattactacggcaaacttcaacgatttctatgatgcattttataattagtaagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaaaggaatttcgacggatcaataacagtgtttgtggagcattttctgaatacaataaacccaaaacagaaacttcccttttgtatcactgttctggaaaaggggtgggcggtaataaagctaatagggtgtgtccataagtaatactgaacttggaaatgtgcggctttgcagcattttgtctttctataaaaatgtgtcgttcctttttttcattttttggcgcgtcgcctcggggtcgtatagaatatgcgtcacttttaaaaataagattgcagatcagggcaaaacaagtagcaaatcatagcaagagaccctgatttttgtgacataaatatttttacttctgtgttaggttaactttttatgtaactgtaaatggaatagagttgaggggatagtgcccacaagtcaatatgtttattttgtaaagttgaaagataattatttttatgctcaggtgattttggtgttgaattttctgtaatattaacataagagtaatacattgagtggttagtatatggtgtaaaagtggtataacgcatgtattaagagcagttatacaatatttggggccgctgaatgagatatagatattaaaatgtggataatcatgggctttatgggtaaatggaacagggtatagaccactgaggcaagtgccgtgcataatgatatgagtgcatctagtggcgaacgtggcgagaaaggaagggaagaaagcgagtgccatctgtgcagacaaacgcatcaggatgccggcccgcggaa

SEQ ID NO: 4 - DNA, 12783 bp, Artificial Sequence
  nucleotide sequence of vector pDB1371
ccatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgaaaattcaaggaatttcgacggatcaataacagtgtttgtggagcattttctgaatacaataaacccaaaacagaaacttcccttttgtatcactgttctggaaaaggggtgggcggtaataaagctaatagggtgtgtccataagtaatactgaacttggaaatgtgcggctttgcagcattttgtctttctataaaaatgtgtcgttcctttttttcattttttggcgcgtcgcctcggggtcgtatagaatatgcgtcacttttaaaaataagattgcagatcagggcaaaacaagtagcaaatcatagcaagagaccctgatttttgtgacataaatatttttacttctgtgttaggttaactttttatgtaactgtaaatggaatagagttgaggggatagtgcccacaagtcaatatgtttattttgtaaagttgaaagataattatttttatgctcaggtgattttggtgttgaattttctgtaatattaacataagagtaatacattgagtggttagtatatggtgtaaaagtggtataacgcatgtattaagagcagttatacaatatttggggccgctgaatgagatatagatattaaaatgtggataatcatgggctttatgggtaaatggaacagggtatagaccactgaggcaagtgccgtgcataatgatatgagtgcatctagtggcgaacgtggcgagaaaggaagggaagaaagcgagtgccatctgtgcagacaaacgcatcaggatgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcataggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgtctattaaatgcttcctatattatatatatagtaatgtcgtttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgcataggccactagtggatctgattcgaattctaccgttcgtatagcatacattatacgaagttatgagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctataacttcgtatagcatacattatacgaacggtagaattcgtcgacctgcagcgtacgaagcttcagctggcggccgcaacaatagcgatccgaaaggcggcaataggtctagaaacttgttcaagttcttagagactatatggcgtacagaaggtcttgcggccctctacacgggcctggcagccagagtaattaagatagcgccaagttgcgccatcatgatatctagttatgagatctccaaaaaagtatttggaaacaaattgcatcagtgaataaaggcttgtaaatatagatatatagtaaacgaaaagaagcatatacgtataattatttgtgggaacggctctagaaaagaaaactttgcctttaactcctttacacctttctcttcttctttggatcagctctggaatcaccacccaattgagacaagtcaattctggtttcgtacaaaccggtaatagattggtgaatcaaagtagcatctaaaacttctttggtggaagtgtatctctttctgtcaatggtagtgtcgaagtatttgaaagcagctggagcacccaagttggttaaagtgaacaaatgaatgatgttttcagcttgttctctgattggtttgtctctgtgcttgttgtaagcggataaaaccttatctaagttagcatcagccaaaatgactctcttggagaattcggagatttgttcaataatttcatccaagtagtgcttgtgttgttcaacaaacaattgcttttgttcgttgtcttctggggaacctttcaacttttcgtagtgggaagccaagtacaagaagttaacgtacttagatggtaaagccaattcgttacctttttgtagttcaccagcggaggccaacattctctttctaccgttttccaattcgaacaaggagtattttggtaacttaatgatcaaatccttcttgacttccttgtaacccttagcttccaaaaagtcgattgggttcttttcgaaggaggatctttccatgatggtgatacccaacaattccttaacagacttcaacttcttagacttacccttttcaaccttagcaacgaccaaaacggagtaagcaacagttggagagtcgaaaccaccgtacttcttaggatcccagtccttctttctagcaatcaacttgtcagagtttctctttggcaaaatggattccttagagaaaccaccagtttgaacttcagtcttcttgacgatgttaacttgtggcatagacaaaacctttctgacggtagcgaaatctctacccttgtcccagacaatttcaccagtttcaccattggtttcaatcaatggacgctttctaatttcaccgttagctaaagtgatttcagtcttgaaaaagttcatgatgttagagtagaagaagtacttagcagtggccttaccaatttcttgttcagacttggcgatcatctttctaacatcgtaaaccttgtagtcaccgtaaacgaattcagattccaactttgggtactttttgattaaggcagtaccgacaacagcgttcaagtaggcatcgtgagcgtgatggtagttgttgatttctctgaccttgtaaaattggaagtcctttctgaagtcagaaaccaacttagacttcaaagtgatgaccttaacttctctaattagtttgtcgttttcatcgtacttagtgttcattctggaatccaagatttgagcaacatgcttggtgatttgtctagtttcgactaattgtctcttgatgaaaccggctttgtccaattcggacaaaccacctctttcagccttggtcaagttgtcgaactttctttgagtgatcaacttagcattcaacaattgtctccagtagttcttcatcttcttaacaacttcttcagatggaacgttatcagacttacctctgttcttgtcagatctagtcaaaactttgttgtcaatggaatcgtccttcaagaacgattgtgggacgatatgatcgacatcgtagtcagacaatctgttgatatccaattcttggtcgacgtacatgtcacgaccgttttgcaagtagtacaagtatagcttttcgttttgtaattgagtgttttcgactgggtgttctttcaaaatttgagaacccaactccttgataccttcttcaattctcttcatacgttctctagagttcttttgacccttttgagtagtttggttttctctagccatttcgatgacaatattttctggcttgtgtctacccatgactttgaccaattcatcaacgaccttgacggtttgtaagatacccttcttaatagctggagaaccagccaagttagcgatgtgttcgtgcaaagaatcaccttgaccagagacttgggctttttgaatgtcttccttgaaagtcaaagaatcgtcgtgaatcaattgcataaagtttctgttagcgaaaccatcggatttcaaaaagtctaaaatagtcttaccggattgcttgtctctgataccgttaatcaactttctggacaatctaccccaaccagtgtatcttcttctctttagttgcttcataactttatcgtcgaacaagtgagcgtaggtcttcaatctctcttcaatcatttctctgtcctcgaaaagagtcaaggtcaaaacgatatcttccaagatgtcttcgttttcttcgttatctaaaaagtccttgtccttgatgatctttaacaaatcgtggtaggtgcccaaagaagcattgaaacggtcttcaacaccggagatttcgacggaatcgaaacattcaatcttcttgaagtagtcttccttcaattgcttaacagtgacctttctgttggtcttaaacaacaagtcaacaatagccttcttttgttcaccggataggaaagctggctttctcataccttcagtaacgtatttaaccttggttaattcgttgtagacggtgaagtattcgtacaacaaagagtgctttggcaagaccttctcgtttggcaagttcttatcaaagttggtcattctttcgatgaaagattgggcagaagcacccttgtcgacgacttcttcgaagttccatggggtgatggtttcttcagactttctggtcatccaagcgaatctagaattacctctggccaatggaccgacgtagtatgggattctgaaagttaagatcttttcgatcttttctctgttgtcctttaggaatggatagaaatcttcctgtcttctcaaaatggcgtgcaattcacccaagtggatttggtgtgggatagaaccgttatcgaaggtacgttgctttctcaataagtcttctctgttcaacttaaccaataattcttcagtaccatccatcttttccaaaattggcttgatgaacttgtagaattcttcctgagaagcaccaccgtcaatgtaaccggcgtaaccatttttggattggtcgaagaagatttccttgtacttttctggcaattgttgtctaaccaaagccttcaacaaagtcagatcttggtggtgttcgtcgtatcttttgatcatagaagcagacaatggagccttggtaatttcagtgttaactctcaagatgtcagacaacaagatagcgtcagataagtttttggcagccaagaacaagtcggcgtattggtcaccgatttgagccaacaagttgtctaagtcgtcgtcgtaggtgtccttggacaattgcaacttggcatcttcagccaagtcgaagttggacttgaagtttggggtcaaacccaaggacaaagcgatcaagttaccgaacaaaccgtttttcttttcaccaggcaattgagcaatcaagttttccaaacgtctagacttggacaaacgggcagataagatggccttagcatcaacaccagaagcgttaattgggttttcctcgaataattggttgtaggtttggaccaattggatgaacaatttgtcgacgtcagagttgtctgggttcaagtcaccttcaatcaagaagtgacctctgaacttgatcatgtgagccaaggccaaatagatcaatctcaaatcagccttgtcggtggaatcgaccaacttctttctcaaatggtagatggtagggtatttttcgtggtaagcaacttcgtcaacgatgttaccgaagattggatgtctttcgtgcttcttgtcttcttcaaccaagaaagattcttccaatctgtggaagaaagagtcgtcaaccttggccatttcgttagaaaagatttcttgcaagtaacaaatacggtttttacgtctagtgtatctacgacgagcggtacgcttcaatctggtagcttcagcggtttcaccggagtcgaacaacaaagcaccgatcaaattcttcttgatagagtgtctgtcagtgttacccaagaccttgaatttcttggatggaaccttgtattcgtcggtgatgacagcccaaccgacggagttggtcccgatatccaaaccaatagagtatttcttgtccatttttgataagtatttaagcgagtgactgaagaataatattctatgaggttttaagctaaaaatgaatatagtaaaattattataataatagtgtagaagaagagaaaagaatatattaaaatctgaatggtagctgctgtatatatactcttttttttgcctcctccttgggtaagtttctctatttcagtagataaaaaaaaaacaataaagcagtaatattttttatcatatctcctcataacaggaaaaatcagaaactaaggtttctaaatttgtcatttttttggccacgccccagtggccgcagtacaaatcgcgagatttgtttgtttttggtcatgaaagaaaaaaatgaaaaataagaaactaaaccaaaaaaaaaaaaacgccaacagtgagaacgggctttacaacggacttttacagtgggttctaaaagaggtaaaaactagcccgtagccagcagactggtattttgattacctgtcttagcgttgactggttgcttattctactagaccgcgtggtagcagacaataatgggagaatacgactcgtttttgcccctttcgtagtcaatcctctcagttcctcttcctcgaaagtagaaacgcagcaactttctcattggcgagtattttggtttttgtttttttgtttcggtttccacgtataatgagtggagttttcggtttgttgaaccgtgtttgctttgctagtagaaacttaccaaattttgaaaaaaatttgacaggctaaggctagcttacctgaggctagctagggtaagcaagattttgctaggatttccacctatggcggatttctcgttctactccgtagtgcttacatttctgagtcgttaacagcgcttgccctcaaatctaaaaaatctagaaaaagagttcctagactaatttggataaatagtaggattcgttgctctacctactcctctgcctccccgccacatgggggtgaccgcaaaaaaagaaaaggtac

SEQ ID NO: 5 - DNA, 12777 bp, Artificial Sequence
  nucleotide sequence of vector pDB1372
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcatcctgatgcgtttgtctgcacagatggcactcgctttcttcccttcctttctcgccacgttcgccactagatgcactcatatcattatgcacggcacttgcctcagtggtctataccctgttccatttacccataaagcccatgattatccacattttaatatctatatctcattcagcggccccaaatattgtataactgctcttaatacatgcgttataccacttttacaccatatactaaccactcaatgtattactcttatgttaatattacagaaaattcaacaccaaaatcacctgagcataaaaataattatctttcaactttacaaaataaacatattgacttgtgggcactatcccctcaactctattccatttacagttacataaaaagttaacctaacacagaagtaaaaatatttatgtcacaaaaatcagggtctcttgctatgatttgctacttgttttgccctgatctgcaatcttatttttaaaagtgacgcatattctatacgaccccgaggcgacgcgccaaaaaatgaaaaaaaggaacgacacatttttatagaaagacaaaatgctgcaaagccgcacatttccaagttcagtattacttatggacacaccctattagctttattaccgcccaccccttttccagaacagtgatacaaaagggaagtttctgttttgggtttattgtattcagaaaatgctccacaaacactgttattgatccgtcgaaattccttttatgtgatgattgattgattgattgtacagtttgtttttcttaatatctatttcgatgacttctatatgatattgcactaacaagaagatattataatgcaattgatacaagacaaggagttatttgcttctcttttatatgattctgacaatccatattgcgttggtagtcttttttgctggaacggttcagcggaaaagacgcatcgctctttttgcttctagaagaaatgccagcaaaagaatctcttgacagtgactgacagcaaaaatgtctttttctaactagtaacaaggctaagatatcagcctgaaataaagggtggtgaagtaataattaaatcatccgtataaacctatacacatatatgaggaaaaataatacaaaagtgttttaaatacagatacatacatgaacatatgcacgtatagcgcccaaatgtcggtaatgggatcggcttactaattataaaatgcatcatagaaatcgttgaagtttgccgtagtaatacccagattatcagattccaaatccttgtcaataattatactcctttggacaacttctctttccattaaaaaatctgaaatctccttaggtaccttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcagctaccattcagattttaatatattcttttctcttcttctacactattattataataattttactatattcatttttagcttaaaacctcatagaatattattcttcagtcactcgcttaaatacttatcaaaaatggacaagaaatactctattggtttggatatcgggaccaactccgtcggttgggctgtcatcaccgacgaatacaaggttccatccaagaaattcaaggtcttgggtaacactgacagacactctatcaagaagaatttgatcggtgctttgttgttcgactccggtgaaaccgctgaagctaccagattgaagcgtaccgctcgtcgtagatacactagacgtaaaaaccgtatttgttacttgcaagaaatcttttctaacgaaatggccaaggttgacgactctttcttccacagattggaagaatctttcttggttgaagaagacaagaagcacgaaagacatccaatcttcggtaacatcgttgacgaagttgcttaccacgaaaaataccctaccatctaccatttgagaaagaagttggtcgattccaccgacaaggctgatttgagattgatctatttggccttggctcacatgatcaagttcagaggtcacttcttgattgaaggtgacttgaacccagacaactctgacgtcgacaaattgttcatccaattggtccaaacctacaaccaattattcgaggaaaacccaattaacgcttctggtgttgatgctaaggccatcttatctgcccgtttgtccaagtctagacgtttggaaaacttgattgctcaattgcctggtgaaaagaaaaacggtttgttcggtaacttgatcgctttgtccttgggtttgaccccaaacttcaagtccaacttcgacttggctgaagatgccaagttgcaattgtccaaggacacctacgacgacgacttagacaacttgttggctcaaatcggtgaccaatacgccgacttgttcttggctgccaaaaacttatctgacgctatcttgttgtctgacatcttgagagttaacactgaaattaccaaggctccattgtctgcttctatgatcaaaagatacgacgaacaccaccaagatctgactttgttgaaggctttggttagacaacaattgccagaaaagtacaaggaaatcttcttcgaccaatccaaaaatggttacgccggttacattgacggtggtgcttctcaggaagaattctacaagttcatcaagccaattttggaaaagatggatggtactgaagaattattggttaagttgaacagagaagacttattgagaaagcaacgtaccttcgataacggttctatcccacaccaaatccacttgggtgaattgcacgccattttgagaagacaggaagatttctatccattcctaaaggacaacagagaaaagatcgaaaagatcttaactttcagaatcccatactacgtcggtccattggccagaggtaattctagattcgcttggatgaccagaaagtctgaagaaaccatcaccccatggaacttcgaagaagtcgtcgacaagggtgcttctgcccaatctttcatcgaaagaatgaccaactttgataagaacttgccaaacgagaaggtcttgccaaagcactctttgttgtacgaatacttcaccgtctacaacgaattaaccaaggttaaatacgttactgaaggtatgagaaagccagctttcctatccggtgaacaaaagaaggctattgttgacttgttgtttaagaccaacagaaaggtcactgttaagcaattgaaggaagactacttcaagaagattgaatgtttcgattccgtcgaaatctccggtgttgaagaccgtttcaatgcttctttgggcacctaccacgatttgttaaagatcatcaaggacaaggactttttagataacgaagaaaacgaagacatcttggaagatatcgttttgaccttgactcttttcgaggacagagaaatgattgaagagagattgaagacctacgctcacttgttcgacgataaagttatgaagcaactaaagagaagaagatacactggttggggtagattgtccagaaagttgattaacggtatcagagacaagcaatccggtaagactattttagactttttgaaatccgatggtttcgctaacagaaactttatgcaattgattcacgacgattctttgactttcaaggaagacattcaaaaagcccaagtctctggtcaaggtgattctttgcacgaacacatcgctaacttggctggttctccagctattaagaagggtatcttacaaaccgtcaaggtcgttgatgaattggtcaaagtcatgggtagacacaagccagaaaatattgtcatcgaaatggctagagaaaaccaaactactcaaaagggtcaaaagaactctagagaacgtatgaagagaattgaagaaggtatcaaggagttgggttctcaaattttgaaagaacacccagtcgaaaacactcaattacaaaacgaaaagctatacttgtactacttgcaaaacggtcgtgacatgtacgtcgaccaagaattggatatcaacagattgtctgactacgatgtcgatcatatcgtcccacaatcgttcttgaaggacgattccattgacaacaaagttttgactagatctgacaagaacagaggtaagtctgataacgttccatctgaagaagttgttaagaagatgaagaactactggagacaattgttgaatgctaagttgatcactcaaagaaagttcgacaacttgaccaaggctgaaagaggtggtttgtccgaattggacaaagccggtttcatcaagagacaattagtcgaaactagacaaatcaccaagcatgttgctcaaatcttggattccagaatgaacactaagtacgatgaaaacgacaaactaattagagaagttaaggtcatcactttgaagtctaagttggtttctgacttcagaaaggacttccaattttacaaggtcagagaaatcaacaactaccatcacgctcacgatgcctacttgaacgctgttgtcggtactgccttaatcaaaaagtacccaaagttggaatctgaattcgtttacggtgactacaaggtttacgatgttagaaagatgatcgccaagtctgaacaagaaattggtaaggccactgctaagtacttcttctactctaacatcatgaactttttcaagactgaaatcactttagctaacggtgaaattagaaagcgtccattgattgaaaccaatggtgaaactggtgaaattgtctgggacaagggtagagatttcgctaccgtcagaaaggttttgtctatgccacaagttaacatcgtcaagaagactgaagttcaaactggtggtttctctaaggaatccattttgccaaagagaaactctgacaagttgattgctagaaagaaggactgggatcctaagaagtacggtggtttcgactctccaactgttgcttactccgttttggtcgttgctaaggttgaaaagggtaagtctaagaagttgaagtctgttaaggaattgttgggtatcaccatcatggaaagatcctccttcgaaaagaacccaatcgactttttggaagctaagggttacaaggaagtcaagaaggatttgatcattaagttaccaaaatactccttgttcgaattggaaaacggtagaaagagaatgttggcctccgctggtgaactacaaaaaggtaacgaattggctttaccatctaagtacgttaacttcttgtacttggcttcccactacgaaaagttgaaaggttccccagaagacaacgaacaaaagcaattgtttgttgaacaacacaagcactacttggatgaaattattgaacaaatctccgaattctccaagagagtcattttggctgatgctaacttagataaggttttatccgcttacaacaagcacagagacaaaccaatcagagaacaagctgaaaacatcattcatttgttcactttaaccaacttgggtgctccagctgctttcaaatacttcgacactaccattgacagaaagagatacacttccaccaaagaagttttagatgctactttgattcaccaatctattaccggtttgtacgaaaccagaattgacttgtctcaattgggtggtgattccagagctgatccaaagaagaagagaaaggtgtaaaggagttaaaggcaaagttttcttttctagagccgttcccacaaataattatacgtatatgcttcttttcgtttactatatatctatatttacaagcctttattcactgatgcaatttgtttccaaatacttttttggagatctcataactagatatcatgatggcgcaacttggcgctatcttaattactctggctgccaggcccgtgtagagggccgcaagaccttctgtacgccatatagtctctaagaacttgaacaagtttctagacctattgccgcctttcggatcgctattgttgcggccgccagctgaagcttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgaatcagatccactagtggcctatgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 6 - DNA, 5042 bp, Artificial Sequence
  nucleotide sequence of vector pDB1368
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggaacgttgtccaggtttgtatccacgtgtgtccgttccgccaatattccgcgtgcgttttatttctgctgccatccgtaaatgccaggatttgagcgggttacacaatatatctcatattttcggtgtctgggtcattactttactcttggcatccactaaatatattggatcctgctttttaaactggcttccagaaaaaaatcaatggagtgatgcaaactgcctggagtaaaagatgacacaaggcgattgacctacgcatgtatctatctcattttcttacaccttctatttcattctaactctttgatttggaaaacacctaagaaaaaaaaggttgaaatcagttccctgaaattgtccccctacttgactaataaatatataaagacggtaggtattgactgtaattcgtaaatctatacttcttaaacttcttcaaatttacttttttggatagtcttatttttggtttcaataccccaagaacttagtttcaaataaatacacatacaaacaaaatgtcaggtgttaataatacatccgcaaatgagttatctactaccatgtctaactctaactcagcagtaggcgctccctctgttaagactgaacacggtgactctaaaaattcccttaacctagatgccaatgagccacctattgacttacctcaaaaacccctcgccgcatattggactgttatctgtttatgtctaatgattgcatttggtgggtttgtctttggttgggatactggtaccatctctggttttgttaatcaaaccgatttcaaaagaagatttggtcaaatgaaatctgatggtacctattatctttcggacgtccggactggtttgatcgttggtatcttcaatattggttgtgcctttggtgggttaaccttaggacgtctgggtgatatgtatggacgtagaattggtttgatgtgcgtcgttctggtatacatcgttggtattgtgattcaaattgcttctagtgacaaatggtaccaatatttcattggtagaattatctctggtatgggtgtcggtggtattgctgtcctatctccaactttgatttccgaaacagcaccaaaacacattagaggtacctgtgtttctttctatcagttaatgatcactctaggtattttcttaggttactgtaccaactatggtactaaagactactccaattcagttcaatggagagtgcctttgggtttgaactttgccttcgctattttcatgatcgctggtatgctaatggttccagaatctccaagattcttagtcgaaaaaggcagatacgaagacgctaaacgttctttggcaaaatctaacaaagtcaccattgaagatccaagtattgttgctgaaatggatacaattatggccaacgttgaaactgaaagattagccggtaacgcttcttggggtgagttattctccaacaaaggtgctattttacctcgtgtgattatgggtattatgattcaatccttacaacaattaactggtaacaattacttcttctattatggtactactattttcaacgccgtcggtatgaaagattctttccaaacttccatcgttttaggtatagtcacgttcgcatccactttcgtggccttatacactgttgataaatttggtcgtcgtaagtgtctattgggtggttctgcttccatggccatttgttttgttatcttctctactgtcggtgtcacaagcttatatccaaatggtaaagatcaaccatcttccaaggctgccggtaacgtcatgattgtctttacctgtttattcattttcttcttcgctattagttgggccccaattgcctacgttattgttgccgaatcctatcctttgcgtgtcaaaaatcgtgctatggctattgctgttggtgccaactggatttggggtttcttgattggtttcttcactcccttcattacaagtgcaattggattttcatacgggtatgtcttcatgggctgtttggtattttcattcttctacgtgtttttctttgtctgtgaaaccaagggcttaacattagaggaagttaatgaaatgtatgttgaaggtgtcaaaccatggaaatctggtagctggatctcaaaagaaaaaagagtttccgaggaataaatttgatctgtagcctaagtataaaattctacgtatgtatatatttacatgcaattttttctttttccaattcatgcctcagaaagcctgtatgcgaagccacaatcctttccaacagaccatactaagtaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 7 - DNA, 35 bp, Artificial Sequence
  nucleotide sequence of Forward primer for extension PCR to add KpnI restriction site to cFS0017
aaccgcggggtacccatttgaataagaagtaatac

SEQ ID NO: 8 - DNA, 33 bp, Artificial Sequence
  nucleotide sequence of Reverse primer for extension PCR to add NgoMIV restriction site to cFS0017
ttccgcgggccggcatcctgatgcgtttgtctg

SEQ ID NO: 9 - DNA, 516 bp, Artificial Sequence
  nucleotide sequence of the INT70 gRNA gBLOCK
gctatacgaacggtagaattcgatatcagatccactagtggcctatgcggccgccaccgcggtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcggagagaaaggcccgggcgtgttttagagctagaaatagcaagttaaaataaggctagtccgttatcaacttgaaaaagtggcaccgagtcggtggtgctttttttgttttttatgtctccgcggtggagctccagcttttgttccctttagtgagggttaattgcgcgcttggcgtaatcatgg

SEQ ID NO: 10 - DNA, 82 bp, Artificial Sequence
  nucleotide sequence of the forward primer to obtain donor DNA PCR fragment (int70[5′]-conD-HXT11/2-con3-int70[3′]) using pDB1368 as template
gaccggtctaagctcttagaggttctcgcatacccaagtaaaagctaagaccgaagcaaaaacgttgtccaggtttgtatcc

SEQ ID NO: 11 - DNA, 84 bp, Artificial Sequence
  nucleotide sequence of the reverse primer to obtain donor DNA PCR fragment (int70[5′]-conD-HXT11/2-con3-int70[3′]) using pDB1368 as template
tttgtttctttattgtttttatttttacgacattttcccctcgaagaatatttatccgaaacttagtatggtctgttggaaagg

SEQ ID NO: 12 - DNA, 70 bp, Artificial Sequence
  nucleotide sequence of the forward primer to obtain a gRNA-recipient plasmid backbone using pRN1120-RFP-gRNA(A) (SEQ ID NO 1) as template
acgctttccggcatcttccagaccacagtatatccatccgcctcctgttgaggaccggtttatcattatc

SEQ ID NO: 13 - DNA, 24 bp, Artificial Sequence
  set out the nucleotide sequence of the reverse primer to obtain a gRNA-recipient plasmid backbone using pRN1120-RFP-gRNA(A) (SEQ ID NO 1) as template
agcggccgcatgctagctccggat

SEQ ID NO: 14 - DNA, 80 bp, Artificial Sequence
  nucleotide sequence of the forward primer to obtain a guide RNA PCR fragment (gRNA-INT70) using INT70 gBLOCK (SEQ ID NO 9) as template
tcatgtttgacagcttatcatcgataatccggagctagcatgcggccgctgttccgcggtctttgaaaagataatgtatg

SEQ ID NO: 15 - DNA, 74 bp, Artificial Sequence
  nucleotide sequence of the reverse primer to obtain a guide RNA PCR fragment (gRNA-INT70) using INT70 gBLOCK (SEQ ID NO 9) as template
caacaggaggcggatggatatactgtggtctggaagatgccggaaagcgccatttgatggagttccgcggagac

SEQ ID NO: 16 - DNA, 20 bp, Artificial Sequence
  nucleotide sequence of the forward primer to confirm to confirm the correct assembly and integration of the HXT11/2 expression cassettes at the INT70 locus
gtctgcataggagccttctg

SEQ ID NO: 17 - DNA, 20 bp, Artificial Sequence
  nucleotide sequence of the reverse primer to confirm the correct assembly and integration of the HXT11/2 expression cassettes at the INT70 locus
aatttaccactgcccatggg

SEQ ID NO: 18 - DNA, 11742 bp, Artificial Sequence
  nucleotide sequence of vector pCSN061
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcagctaccattcagattttaatatattcttttctcttcttctacactattattataataattttactatattcatttttagcttaaaacctcatagaatattattcttcagtcactcgcttaaatacttatcaaaaatggacaagaaatactctattggtttggatatcgggaccaactccgtcggttgggctgtcatcaccgacgaatacaaggttccatccaagaaattcaaggtcttgggtaacactgacagacactctatcaagaagaatttgatcggtgctttgttgttcgactccggtgaaaccgctgaagctaccagattgaagcgtaccgctcgtcgtagatacactagacgtaaaaaccgtatttgttacttgcaagaaatcttttctaacgaaatggccaaggttgacgactctttcttccacagattggaagaatctttcttggttgaagaagacaagaagcacgaaagacatccaatcttcggtaacatcgttgacgaagttgcttaccacgaaaaataccctaccatctaccatttgagaaagaagttggtcgattccaccgacaaggctgatttgagattgatctatttggccttggctcacatgatcaagttcagaggtcacttcttgattgaaggtgacttgaacccagacaactctgacgtcgacaaattgttcatccaattggtccaaacctacaaccaattattcgaggaaaacccaattaacgcttctggtgttgatgctaaggccatcttatctgcccgtttgtccaagtctagacgtttggaaaacttgattgctcaattgcctggtgaaaagaaaaacggtttgttcggtaacttgatcgctttgtccttgggtttgaccccaaacttcaagtccaacttcgacttggctgaagatgccaagttgcaattgtccaaggacacctacgacgacgacttagacaacttgttggctcaaatcggtgaccaatacgccgacttgttcttggctgccaaaaacttatctgacgctatcttgttgtctgacatcttgagagttaacactgaaattaccaaggctccattgtctgcttctatgatcaaaagatacgacgaacaccaccaagatctgactttgttgaaggctttggttagacaacaattgccagaaaagtacaaggaaatcttcttcgaccaatccaaaaatggttacgccggttacattgacggtggtgcttctcaggaagaattctacaagttcatcaagccaattttggaaaagatggatggtactgaagaattattggttaagttgaacagagaagacttattgagaaagcaacgtaccttcgataacggttctatcccacaccaaatccacttgggtgaattgcacgccattttgagaagacaggaagatttctatccattcctaaaggacaacagagaaaagatcgaaaagatcttaactttcagaatcccatactacgtcggtccattggccagaggtaattctagattcgcttggatgaccagaaagtctgaagaaaccatcaccccatggaacttcgaagaagtcgtcgacaagggtgcttctgcccaatctttcatcgaaagaatgaccaactttgataagaacttgccaaacgagaaggtcttgccaaagcactctttgttgtacgaatacttcaccgtctacaacgaattaaccaaggttaaatacgttactgaaggtatgagaaagccagctttcctatccggtgaacaaaagaaggctattgttgacttgttgtttaagaccaacagaaaggtcactgttaagcaattgaaggaagactacttcaagaagattgaatgtttcgattccgtcgaaatctccggtgttgaagaccgtttcaatgcttctttgggcacctaccacgatttgttaaagatcatcaaggacaaggactttttagataacgaagaaaacgaagacatcttggaagatatcgttttgaccttgactcttttcgaggacagagaaatgattgaagagagattgaagacctacgctcacttgttcgacgataaagttatgaagcaactaaagagaagaagatacactggttggggtagattgtccagaaagttgattaacggtatcagagacaagcaatccggtaagactattttagactttttgaaatccgatggtttcgctaacagaaactttatgcaattgattcacgacgattctttgactttcaaggaagacattcaaaaagcccaagtctctggtcaaggtgattctttgcacgaacacatcgctaacttggctggttctccagctattaagaagggtatcttacaaaccgtcaaggtcgttgatgaattggtcaaagtcatgggtagacacaagccagaaaatattgtcatcgaaatggctagagaaaaccaaactactcaaaagggtcaaaagaactctagagaacgtatgaagagaattgaagaaggtatcaaggagttgggttctcaaattttgaaagaacacccagtcgaaaacactcaattacaaaacgaaaagctatacttgtactacttgcaaaacggtcgtgacatgtacgtcgaccaagaattggatatcaacagattgtctgactacgatgtcgatcatatcgtcccacaatcgttcttgaaggacgattccattgacaacaaagttttgactagatctgacaagaacagaggtaagtctgataacgttccatctgaagaagttgttaagaagatgaagaactactggagacaattgttgaatgctaagttgatcactcaaagaaagttcgacaacttgaccaaggctgaaagaggtggtttgtccgaattggacaaagccggtttcatcaagagacaattagtcgaaactagacaaatcaccaagcatgttgctcaaatcttggattccagaatgaacactaagtacgatgaaaacgacaaactaattagagaagttaaggtcatcactttgaagtctaagttggtttctgacttcagaaaggacttccaattttacaaggtcagagaaatcaacaactaccatcacgctcacgatgcctacttgaacgctgttgtcggtactgccttaatcaaaaagtacccaaagttggaatctgaattcgtttacggtgactacaaggtttacgatgttagaaagatgatcgccaagtctgaacaagaaattggtaaggccactgctaagtacttcttctactctaacatcatgaactttttcaagactgaaatcactttagctaacggtgaaattagaaagcgtccattgattgaaaccaatggtgaaactggtgaaattgtctgggacaagggtagagatttcgctaccgtcagaaaggttttgtctatgccacaagttaacatcgtcaagaagactgaagttcaaactggtggtttctctaaggaatccattttgccaaagagaaactctgacaagttgattgctagaaagaaggactgggatcctaagaagtacggtggtttcgactctccaactgttgcttactccgttttggtcgttgctaaggttgaaaagggtaagtctaagaagttgaagtctgttaaggaattgttgggtatcaccatcatggaaagatcctccttcgaaaagaacccaatcgactttttggaagctaagggttacaaggaagtcaagaaggatttgatcattaagttaccaaaatactccttgttcgaattggaaaacggtagaaagagaatgttggcctccgctggtgaactacaaaaaggtaacgaattggctttaccatctaagtacgttaacttcttgtacttggcttcccactacgaaaagttgaaaggttccccagaagacaacgaacaaaagcaattgtttgttgaacaacacaagcactacttggatgaaattattgaacaaatctccgaattctccaagagagtcattttggctgatgctaacttagataaggttttatccgcttacaacaagcacagagacaaaccaatcagagaacaagctgaaaacatcattcatttgttcactttaaccaacttgggtgctccagctgctttcaaatacttcgacactaccattgacagaaagagatacacttccaccaaagaagttttagatgctactttgattcaccaatctattaccggtttgtacgaaaccagaattgacttgtctcaattgggtggtgattccagagctgatccaaagaagaagagaaaggtgtaaaggagttaaaggcaaagttttcttttctagagccgttcccacaaataattatacgtatatgcttcttttcgtttactatatatctatatttacaagcctttattcactgatgcaatttgtttccaaatacttttttggagatctcataactagatatcatgatggcgcaacttggcgctatcttaattactctggctgccaggcccgtgtagagggccgcaagaccttctgtacgccatatagtctctaagaacttgaacaagtttctagacctattgccgcctttcggatcgctattgttgcggccgccagctgaagcttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgaatcagatccactagtggcctatgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 19 - DNA, 605 bp, Artificial Sequence
  nucleotide sequence of the pGAL10 promoter
catttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgaaaattc

SEQ ID NO: 20 - DNA, 600 bp, Artificial Sequence
  nucleotide sequence of the pCUP1 promoter
ttatgtgatgattgattgattgattgtacagtttgtttttcttaatatctatttcgatgacttctatatgatattgcactaacaagaagatattataatgcaattgatacaagacaaggagttatttgcttctcttttatatgattctgacaatccatattgcgttggtagtcttttttgctggaacggttcagcggaaaagacgcatcgctctttttgcttctagaagaaatgccagcaaaagaatctcttgacagtgactgacagcaaaaatgtctttttctaactagtaacaaggctaagatatcagcctgaaataaagggtggtgaagtaataattaaatcatccgtataaacctatacacatatatgaggaaaaataatacaaaagtgttttaaatacagatacatacatgaacatatgcacgtatagcgcccaaatgtcggtaatgggatcggcttactaattataaaatgcatcatagaaatcgttgaagtttgccgtagtaatacccagattatcagattccaaatccttgtcaataattatactcctttggacaacttctctttccattaaaaaatctgaaatctccttag

SEQ ID NO: 21 - DNA, 758 bp, Artificial Sequence
  GIN11(M86)
aaggaatttcgacggatcaataacagtgtttgtggagcattttctgaatacaataaacccaaaacagaaacttcccttttgtatcactgttctggaaaaggggtgggcggtaataaagctaatagggtgtgtccataagtaatactgaacttggaaatgtgcggctttgcagcattttgtctttctataaaaatgtgtcgttcctttttttcattttttggcgcgtcgcctcggggtcgtatagaatatgcgtcacttttaaaaataagattgcagatcagggcaaaacaagtagcaaatcatagcaagagaccctgatttttgtgacataaatatttttacttctgtgttaggttaactttttatgtaactgtaaatggaatagagttgaggggatagtgcccacaagtcaatatgtttattttgtaaagttgaaagataattatttttatgctcaggtgattttggtgttgaattttctgtaatattaacataagagtaatacattgagtggttagtatatggtgtaaaagtggtataacgcatgtattaagagcagttatacaatatttggggccgctgaatgagatatagatattaaaatgtggataatcatgggctttatgggtaaatggaacagggtatagaccactgaggcaagtgccgtgcataatgatatgagtgcatctagtggcgaacgtggcgagaaaggaagggaagaaagcgagtgccatctgtgcagacaaacgcatcaggat

(19) United States
(12) Patent Application Publication
(10) Pub. No.: US 2020/0392513 A1
(43) Pub. Date: Dec. 17, 2020

(54) A METHOD FOR GENOME EDITING IN A HOST CELL

(71) Applicant: DSM IP ASSETS B.V., Heerlen (NL)

(72) Inventors: Rene VERWAAL, Echt (NL); Paulus Petrus DE WAAL, Echt (NL); Francine Maruschka Johanna DE LEEUW-VAN LOON, Echt (NL)

(21) Appl. No.: 16/955,255

(22) PCT Filed: Nov. 20, 2018
(86) PCT No.: PCT/EP2018/081942

§ 371 Date: Jun. 18, 2020

(30) Foreign Application Priority Data
Dec. 20, 2017 (EP) 17209063.1

(51) Int. Cl. C12N 15/64; C12N 5/10; C12N 15/10; C07K 14/47; C12N 15/85

(52) U.S. Cl. CPC C12N 15/64; C12N 5/10; C12N 2015/8518; C07K 14/4705; C12N 15/10

(57) ABSTRACT

The present invention relates to the field of molecular biology and cell biology. More specifically, the present invention relates to a genome editing system.

### FIELD

The present invention relates to the field of molecular biology and cell biology. More specifically, the present invention relates to a genome editing system.

### BACKGROUND

A polynucleotide-guided nuclease system, also referred to as polynucleotide-guided genome editing system, from which the CRISPR/Cas9 system is a well-known example, is a powerful tool that has been leveraged for genome editing. This tool requires at least a polynucleotide-guided nuclease (polynucleotide-guided genome editing enzyme) such as Cas9 and a guide-polynucleotide such as a guide-RNA that enables the genome editing enzyme to target a specific sequence of DNA. In addition, for editing of the genome in a precise way, a donor polynucleotide such as a donor DNA might be required, especially when relying on homologous recombination for precise genome editing at a desired spot in genomic DNA instead of relying on repair by a random repair process, such as non-homologous end joining.

Several of these required features may be introduced into the cell on an (episomal) expression construct. After the desired genome editing has been performed, it is preferred, especially before industrial scale fermentations that such (episomal) expression construct is removed from the edited cell. Counter-selection using methods known in the art is not always very efficient or expedient since these methods can be time-consuming and have varying efficiencies (from 0% to 100%), making it sometimes necessary to repeat the cycle of time-consuming removal of the (episomal) expression constructs. Accordingly, there is a need for an improved system to remove such (episomal) expression constructs from a host cell after a step of genome editing.

### BRIEF DESCRIPTION OF THE DRAWINGS

FIG. 1 depicts the strategy for integration of the HXT11/2 expression cassette (SparTDH3p-HXT11/2N366T-EFM1t) at the INT70 locus. 5′ and 3′ of part of the donor DNA represent homology of the donor DNA with the INT70 locus, d and 3 represent 50 bp synthetic DNA connector sequences.

FIG. 2 depicts a picture of an agarose gel to confirm integration of the HXT11/2 expression cassettes at the INT70 locus by analysis of PCR fragments.

FIG. 3 depicts the efficiency of loss of plasmid pDB1371. After 2 days of growth at 30° C., 20 colonies per condition were streaked to YEPhD-G418 and YEPhD to score the efficiency of plasmid loss. Different incubation times on YEPhD or YEPhG liquid medium are indicated on the X-axis. The Y-axis represented the number of colonies able to grow on YEPhD-G418 plates (not having lost plasmid pDB1371), out of 20 colonies per growth condition that were initially streaked.

FIG. 4 depicts the efficiency of plasmid loss of plasmid pDB1371 (CP-71-HXT) and pCSN061 (CP-61-HXT). After 2 days of growth at 30° C. on YEPhG agar plates, 40 colonies were streaked to YEPhD-G418 and YEPhD to determine the efficiency of loss of the Cas9-containing plasmid. The Y-axis represented the number of colonies able to grow on YEPhD-G418 plates (not having lost plasmid pDB1371), out of 40 colonies that were initially streaked.

FIG. 5 depicts the efficiency of plasmid loss by inducing the CUP1p-GIN11(M86) construct present on pDB1372. After 2 days of growth at 30° C., the number of colonies were counted per condition to score the relative efficiency of plasmid loss compared to the no-induction condition. The Y-axis represented the percentage of colonies able to grow on YEPhD-G418 plates (not having lost plasmid pDB1372).

FIG. 6 depicts the vector map of single copy (CEN/ARS) vector pCSN061 expressing CAS9 codon pair optimized for expression in S. cerevisiae (SEQ ID NO: 18). A KanMX marker is present on the vector.

FIG. 7 depicts the vector map of pRN1120-RFP-gRNA(A), a natMX marker-containing shuttle vector based on pRS305 with 2-micron origin and expression cassette TDH3p-RFP-PGI1t.

FIG. 8 depicts the vector map of pDB1371, pCSN061 containing the pGAL10-GIN11(M86) polynucleotide sequence.

FIG. 9 depicts the vector map of pDB1372, pCSN061 containing the pCUP1-GIN11(M86) nucleotide sequence.

FIG. 10 depicts the vector map of pDB1368, cloning vector with expression cassette SparTDH3p-HXT11/2N366T-EFM1t.

### DESCRIPTION OF THE SEQUENCES

SEQ ID NO: 1 set out the nucleotide sequence of vector pRN1120-RFP-gRNA(A).

SEQ ID NO: 2 set out the nucleotide sequence of synthetic expression cassette cFS0017 (pGAL10-GIN11(M86)).

SEQ ID NO: 3 set out the nucleotide sequence of synthetic expression cassette cFS0018 (pCUP1-GIN11(M86)).

SEQ ID NO: 4 set out the nucleotide sequence of vector pDB1371.

SEQ ID NO: 5 set out the nucleotide sequence of vector pDB1372.

SEQ ID NO: 6 set out the nucleotide sequence of vector pDB1368.

SEQ ID NO: 7 set out the nucleotide sequence of Forward primer for extension PCR to add KpnI restriction site to cFS0017.

SEQ ID NO: 8 set out the nucleotide sequence of Reverse primer for extension PCR to add NgoMIV restriction site to cFS0017.

SEQ ID NO: 9 set out the nucleotide sequence of the INT70 gRNA gBLOCK.

SEQ ID NO: 10 set out the nucleotide sequence of the forward primer to obtain donor DNA PCR fragment (int70[5′]-conD-HXT11/2-con3-int70[3′]) using pDB1368 as template.

SEQ ID NO: 11 set out the nucleotide sequence of the reverse primer to obtain donor DNA PCR fragment (int70[5′]-conD-HXT11/2-con3-int70[3′]) using pDB1368 as template.

SEQ ID NO: 12 set out the nucleotide sequence of the forward primer to obtain a gRNA-recipient plasmid backbone using pRN1120-RFP-gRNA(A) (SEQ ID NO: 1) as template.

SEQ ID NO: 13 set out the nucleotide sequence of the reverse primer to obtain a gRNA-recipient plasmid backbone using pRN1120-RFP-gRNA(A) (SEQ ID NO: 1) as template.

SEQ ID NO: 14 set out the nucleotide sequence of the forward primer to obtain a guide RNA PCR fragment (gRNA-INT70) using INT70 gBLOCK (SEQ ID NO: 9) as template.

SEQ ID NO: 15 set out the nucleotide sequence of the reverse primer to obtain a guide RNA PCR fragment (gRNA-INT70) using INT70 gBLOCK (SEQ ID NO: 9) as template.

SEQ ID NO: 16 set out the nucleotide sequence of the forward primer to confirm to confirm the correct assembly and integration of the HXT11/2 expression cassettes at the INT70 locus.

SEQ ID NO: 17 set out the nucleotide sequence of the reverse primer to confirm the correct assembly and integration of the HXT11/2 expression cassettes at the INT70 locus.

SEQ ID NO: 18 set out the nucleotide sequence of vector pCSN061.

SEQ ID NO: 19 sets out the nucleotide sequence of the pGAL10 promoter.

SEQ ID NO: 20 sets out the nucleotide sequence of the pCUP1 promoter.

SEQ ID NO: 21 set out the nucleotide sequence of GIN11(M86).

### DETAILED DESCRIPTION

The inventors have found that an effective method of active selection against plasmids containing a Cas9 expression cassette using the growth inhibitory sequence GIN11(M86) (Akada et al., Yeast, vol. 19, pp. 393-402, 2002). Overexpression of this polynucleotide sequence leads to a strong growth-inhibitory effect. GIN11 is a part of the conserved subtelomeric X-element, which is important during chromosomal replication. Since GIN11 was previously found to contain a conserved autonomously replicating sequence (ARS) which may hinder chromosomal integration (Kawahata et al., Yeast, vol. 15, no. 1, pp. 1-10, 1999), a mutant sequence was isolated that lost the replication activity, but retained the growth-inhibitory effect when overexpressed: GIN11(M86). As the polynucleotide sequence does not encode a protein, there is a decreased chance on mutants that lose their growth-inhibitory effect. Coupled to an inducible promoter, episomally expressed plasmids and integrative constructs bearing an inducible GIN11(M86) sequence show efficient gene loss. This method can conveniently be used in a method for genome editing.

Accordingly, in a first aspect, the present invention relates to a method for genome editing in a host cell comprising:

a) contacting a host cell with:

i) an expression construct comprising a polynucleotide that has a negative influence on the viability of the host cell when expressed, operably linked to an inducible promoter,

ii) a functional heterologous genome editing enzyme, or an expression construct capable of expressing a functional heterologous genome editing enzyme in the host cell,

(iii) a guide-polynucleotide, or an expression construct capable of expressing a guide-polynucleotide in the host cell, and, optionally,

(iv) an exogenous polynucleotide,

b) culturing the host cell under conditions that induce genome editing, and

c) culturing the host cell under conditions that induce the expression of the polynucleotide that has a negative influence on the viability of the host cell;

wherein at least an expression construct capable of expressing the functional heterologous genome editing enzyme in the host cell or an expression construct capable of expressing the guide-polynucleotide in the host cell is located on the expression construct comprising the polynucleotide that has a negative influence on the viability of the host cell when expressed.

The method for genome editing, the host cell, the expression construct comprising a polynucleotide that has a negative influence on the viability of the host cell when expressed and the inducible promoter are herein referred to as the method for genome editing according to the invention, the host cell according to the invention, the expression construct comprising a polynucleotide that has a negative influence on the viability of the host cell when expressed according to the invention and the inducible promoter according to the invention.

The basics of the method are that the genome editing process is performed and that after genome editing has taken place and optionally selection of a cell wherein the desired genome editing has taken place, the cell is or the cells are cultured under conditions that induce expression of the polynucleotide that has a negative influence on the viability of the host cell, and the cell subsequently loses the expression construct (e.g. a plasmid) that carries the polynucleotide that has a negative influence on the viability of the host cell. Before the expression construct that carries the polynucleotide that has a negative influence on the viability of the host cell is lost, it may be present episomally or may be integrated in the genome of the host cell.

The polynucleotide that has a negative influence on the viability of the host cell when expressed may be any polynucleotide that has such effect on a host cell. The person skilled in the art knows how to identify such polynucleotide or how to adapt it. The person skilled in the art can for instance use the sequence of the growth inhibitory polynucleotide GIN11(M86) (SEQ ID NO: 21) and adapt it for use in other organisms than yeast. Dependent which expression construct is desired to be lost, the polynucleotide that has a negative influence on the viability of the host cell when expressed can be located on the expression construct capable of expressing the functional heterologous genome editing enzyme in the host cell or the expression construct capable of expressing the guide-polynucleotide. The polynucleotide that has a negative influence on the viability of the host cell when expressed can be also present on both an expression construct capable of expressing the functional heterologous genome editing enzyme in the host cell and on an expression construct capable of expressing the guide-polynucleotide when expressed. Possibly, a polynucleotide encoding a functional heterologous genome editing enzyme and a polynucleotide encoding a guide-polynucleotide of a part thereof, may be present on a single expression construct. In such case, the polynucleotide that has a negative influence on the viability of the host cell when expressed, will be present on this single construct.

The polynucleotide that has a negative influence on the viability of the host cell when expressed is selectively expressed, i.e. when the host cell is cultured under conditions that induce the expression of the polynucleotide that has a negative influence on the viability of the host cell. Such selective expression is known to the person skilled in the art, e.g. a promoter can be used that is only active under selective conditions or a selective transcription factor can be used.

Optionally, in the method according to the invention, an exogenous polypeptide is present. Such polypeptide is typically a donor polynucleotide that is to be introduced during the genome editing step into an acceptor polynucleotide such as the genome of the host cell.

The method according to the invention may be performed as a single method, i.e. performing steps (a) to (c) consecutively or the steps may be performed individually with a pause between steps. Some additional steps may be introduced, such as e.g. the selection of a cell of interest wherein the desired genome editing has taken place after step (b) and before step (c), or after step (c). Negative influence on the viability is herein to be construed as that the host cell, when the polynucleotide that has a negative influence on the viability of the host cell is expressed is less viable than a cell wherein the polynucleotide that has a negative influence on the viability of the host cell is not expressed. Preferably, the growth of the host cell is impaired when the polynucleotide that has a negative influence on the viability of the host cell is expressed. As a consequence, the host cell has an advantage when the polynucleotide (including the construct that carries the polynucleotide) that has a negative influence on the viability of the host cell is lost from the host cell.

Contacting the host cell according to the invention with a construct and/or polynucleotide according to the invention may be performed in any way known to the person skilled in the art, such as, but not limited to, transfection or transformation of cells or parts of cells (such as protoplasts). It will be comprehended by the person skilled in the art that contacting the host cell according to the invention with a construct and/or polynucleotide according to the invention preferably results and thus preferably is equivalent to introduction of a construct and/or polynucleotide according to the invention into the host cell according to the invention.

A guide-polynucleotide according to the invention may any guide-polynucleotide known to the person skilled in the art. Such guide-polynucleotide may be a DNA or an RNA. A guide-polynucleotide according to the present invention comprises at least a guide-sequence that is able to hybridize with a target-polynucleotide and is able to direct sequence-specific binding of the heterologous genome editing system to the target-polynucleotide. The guide-polynucleotide is a polynucleotide according to the general definition of a polynucleotide set out here above; a preferred guide-polynucleotide comprises ribonucleotides, a more preferred guide-polynucleotide is an RNA (guide-RNA). A guide-RNA typically comprises a guide-sequence (crRNA) and a guide-polynucleotide structural component (see e.g. DiCarlo et al., Genome engineering in Saccharomyces cerevisiae using CRISPR-Cas systems. Nucleic Acids Res. 2013; 41(7):4336-4). The guide-sequence is herein also referred as the target sequence and is essentially the complement of a target-polynucleotide such that the guide-polynucleotide is able to hybridize with the target-polynucleotide, preferably under physiological conditions in a host cell.

The functional heterologous genome editing enzyme according to the invention may be any suitable functional genome editing enzyme for use in all embodiments of the invention known to the person skilled in the art and include, but are not limited to: Transcription Activator-Like Effector Nucleases (TALENs, Gaj et al., Trends in Biotechnology, 2013, Vol. 31, No. 7 397-405), zinc finger nucleases (ZFNs, Gaj et al., Trends in Biotechnology, 2013, Vol. 31, No. 7 397-405), meganucleases such as I-Scel (Cabaniols and Paques. Methods Mol Biol. 2008; 435:31-45), RNA-guided endonucleases like CRISPR/Cas (Mali et al., Science. 2013 Feb. 15; 339(6121):823-6; Cong et al., Science. 2013 Feb. 15; 339(6121):819-23), CRISPR/Cpf1 (Zetsche et al., Cell. 2015 Oct. 22; 163(3):759-71) or Cas9 orthologs (reviewed by Mitsunobu et al., Trends Biotechnol. 2017 October; 35(10):983-996), engineered Cas9s with modified properties, e.g. nickase, nuclease dead Cas9 (dCas9) or Cas9 with a modified PAM preference (reviewed by Mitsunobu et al., Trends Biotechnol. 2017 October; 35(10):983-996), dCas9-based transcriptional activators or repressors (reviewed by Mitsunobu et al., Trends Biotechnol. 2017 October; 35(10):983-996), deaminase-mediated base editors (Komor et al., Nature. 2016 May 19; 533(7603):420-424); reviewed by Hess et al., Mol Cell. 2017 Oct. 5; 68(1):26-43; Gaudelli et al., Nature. 2017 Nov. 23; 551(7681):464-471) or CRISPR systems used to introduce epigenetic modifications like histone acetylation, deacetylation or demethylation, or DNA methylation/demethylation (reviewed by Montalbano et al., Mol Cell. 2017 Oct. 5; 68(1):44-59). Functional genome editing systems are known to the person skilled in the art and the person skilled in the art knows how to select and use an appropriate system. A preferred functional genome editing system is an RNA- or DNA-guided nuclease system, preferably an RNA- or DNA-guided DNA nuclease system, more preferably an RNA- or DNA-guided DNA nuclease system that is Protospacer Adjacent Motif (PAM) independent.

In the method according to the invention, at least an expression construct capable of expressing the functional heterologous genome editing enzyme in the host cell or an expression construct capable of expressing the guide-polynucleotide in the host cell, is located on the expression construct comprising the polynucleotide that has a negative influence on the viability of the host cell when expressed. Either the guide-polynucleotide or the genome editing enzyme may be provided as such. However, one of these should always be provided by an expression construct encoding it. The person skilled in the art will comprehend that more than one expression construct may carry comprising the polynucleotide that has a negative influence on the viability of the host cell when expressed; e.g. both the expression construct capable of expressing a functional heterologous genome editing enzyme in the host cell and an expression construct capable of expressing a guide-polynucleotide in the host cell may carry the polynucleotide that has a negative influence on the viability of the host cell when expressed. Other embodiments are possible as well, such as iterative use of the method according to the invention or use of the polynucleotide that has a negative influence on the viability of the host cell when expressed on a library of expression constructs encoding a guide-polynucleotide. The person skilled in the art will comprehend the multiple and multiplex options of the method according to the invention.

Preferably, in a method according to the invention, a prokaryotic host cell, a eukaryotic host cell, a marine eukaryote, a microalgae, a protist or an algae host cell.

Preferably, in a method according to the invention, the host cell is a eukaryotic host cell and preferably is fungal host cell, more preferably a yeast or a filamentous fungal host cell.

Preferably, in a method according to the invention, the host cell is a yeast cell and preferably is a Saccharomyces host cell, preferably a Saccharomyces cerevisiae host cell.

Preferred host cells according to the invention are listed in the section “General Definitions”.

Preferably, in a method according to the invention, the expression construct the expression construct comprising the polynucleotide that has a negative influence on the viability of the host cell when expressed, is present on an episomal entity which is preferably a plasmid.

Preferably, in a method according to the invention, the genome editing enzyme is a Cas-like enzyme. A Cas-like enzyme is construed a polynucleotide-guided endonuclease, such as but not limited to RNA-guided endonucleases like CRISPR/Cas (Mali et al., 2013; Cong et al., 2013) or CRISPR/Cpf1 (Zetsche et al., 2015).

In a method according to the invention, the inducible promoter can be any suitable inducible promoter known to the person skilled in the art. Such inducible promoter, may be a nutrient- (e.g. ammonia, glucose, galactose), metal-, pH- or light-dependent promoter. An inducible promoter may be regulated by an activator and/or repressor, in either cis- or trans-mode. Preferably, in a method according to the invention, the inducible promoter is a copper inducible promoter, preferably a CUP1 promoter or a galactose inducible promoter, preferably a GAL10 promoter. When the promoter is a CUP1 promoter, the CUP1 promoter has preferably at least 80% sequence identity with SEQ ID NO: 20. More preferably, the CUP1 promoter has at least 81, 82, 83, 84, 85, 86, 87, 88, 89, 90, 91, 92, 93, 94, 95, 96, 97, 98, or at least 99% sequence identity with SEQ ID NO: 20, Most preferably, the CUP1 promoter comprises or consists of SEQ ID NO: 20. When the promoter is a GAL10 promoter, the GAL10 promoter preferably has at least 80% sequence identity with SEQ ID NO: 19. More preferably, the GAL10 promoter has at least 81, 82, 83, 84, 85, 86, 87, 88, 89, 90, 91, 92, 93, 94, 95, 96, 97, 98, or at least 99% sequence identity with SEQ ID NO: 19.

Preferably, in a method according to the invention, the polynucleotide that has a negative influence on the viability of the host cell when expressed has at least 80% sequence identity with SEQ ID NO: 21.

In a second aspect, the invention provides for a host cell obtainable by or obtained by a method according to the invention. The features of this second aspect are preferably those of the first aspect of the invention.

In a third aspect, the invention provides for a method for the production of a compound of interest comprising culturing a host cell according to the second aspect of the invention under conditions conducive to the expression of the compound of interest and, optionally, isolating and/or purifying the compound of interest. The compound of interest may be any compound of interest and is preferably one as presented in the section “General Definitions”.

In a fourth aspect, the invention provides for a method for the production of a compound of interest comprising performing the method according to the first aspect of the invention and subsequently culturing said host cell under conditions conducive to the expression of the compound of interest and, optionally, isolating and/or purifying the compound of interest.

### General Definitions

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The terms “a” and “an” are used herein to refer to one or to more than one (i.e. to one or at least one) of the grammatical object of the article. By way of example, “an element” may mean one element or more than one element.

The word “about” or “approximately” when used in association with a numerical value (e.g. about 10) preferably means that the value may be the given value (of 10) more or less 1% of the value. A polynucleotide refers herein to a polymeric form of nucleotides of any length or a defined specific length-range or length, of either deoxyribonucleotides or ribonucleotides, or mixes or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, constructs, vectors, isolated DNA of any sequence, isolated RNA of any sequence, nucleic acid probes, oligonucleotides and primers. A polynucleotide may comprise natural and non-natural nucleotides and may comprise one or more modified nucleotides, such as a methylated nucleotide and a nucleotide analogue or nucleotide equivalent wherein a nucleotide analogue or equivalent is defined as a residue having a modified base, and/or a modified backbone, and/or a non-natural internucleoside linkage, or a combination of these modifications. As desired, modifications to the nucleotide structure may be introduced before or after assembly of the polynucleotide. A polynucleotide may be further modified after polymerization, such as by conjugation with a labeling compound.

In general, codon optimization refers to a process of modifying a nucleic acid sequence for enhanced expression in a host cell of interest by replacing at least one codon (e.g. more than 1, 2, 3, 4, 5, 10, 15, 20, 25, 50, or more codons) of a native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See e.g. Nakamura, Y., et al., 2000. Computer algorithms for codon optimizing a particular sequence for expression in a particular host cell are also available, such as Gene Forge (Aptagen; Jacobus, Pa.), are also available. Preferably, one or more codons (e.g. 1, 2, 3, 4, 5, 10, 15, 20, 25, 50, or more, or all codons) in a sequence encoding a Cas protein correspond to the most frequently used codon for a particular amino acid. Preferred methods for codon optimization are described in WO2006/077258 and WO2008/000632). WO2008/000632 addresses codon-pair optimization. Codon-pair optimization is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence.

In an RNA molecule with a 5′-cap, a 7-methylguanylate residue is located on the 5′ terminus of the RNA (such as typically in mRNA in eukaryotes). RNA polymerase II (Pol II) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase. The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export.

A ribozyme refers to one or more RNA sequences that form secondary, tertiary, and/or quaternary structure(s) that can cleave RNA at a specific site. A ribozyme includes a “self-cleaving ribozyme, or self-processing ribozyme” that is capable of cleaving RNA at a c/s-site relative to the ribozyme sequence (i.e., auto-catalytic, or self-cleaving). The general nature of ribozyme nucleolytic activity is known to the person skilled in the art. The use of self-processing ribozymes in the production of guide-RNA's for RNA-guided nuclease systems such as CRISPR/Cas is inter alia described by Gao et al, Integr Plant Biol. 2014 April; 56(4):343-9.

A nucleotide analogue or equivalent typically comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. It is further preferred that the linkage between a residue in a backbone does not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen et al., 1991. Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar, 2005. Chem. Commun, 495-497). Since the backbone of a PNA molecule contains no charged phosphate groups, PNA-RNA hybrids are usually more stable than RNA-RNA or RNA-DNA hybrids, respectively (Egholm et al., 1993. Nature 365, 566-568).

A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

A further preferred nucleotide analogue or equivalent comprises a substitution of at least one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; aminoxy, methoxyethoxy; -dimethylaminooxyethoxy; and -dimethylaminoethoxyethoxy. The sugar moiety can be a pyranose or derivative thereof, or a deoxypyranose or derivative thereof, preferably a ribose or a derivative thereof, or deoxyribose or derivative thereof. Such preferred derivatized sugar moieties comprise Locked Nucleic Acid (LNA), in which the 2′-carbon atom is linked to the 3′ or 4′ carbon atom of the sugar ring thereby forming a bicyclic sugar moiety. A preferred LNA comprises 2′-0,4′-C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target.

“Sequence identity” or “identity” in the context of the invention of an amino acid- or nucleic acid-sequence is herein defined as a relationship between two or more amino acid (peptide, polypeptide, or protein) sequences or two or more nucleic acid (nucleotide, oligonucleotide, polynucleotide) sequences, as determined by comparing the sequences. In the art, “identity” also means the degree of sequence relatedness between amino acid or nucleotide sequences, as the case may be, as determined by the match between strings of such sequences. Within the invention, sequence identity with a particular sequence preferably means sequence identity over the entire length of said particular polypeptide or polynucleotide sequence.

“Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole sequence (SEQ ID NO:) as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons. Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A polynucleotide according to the invention is represented by a nucleotide sequence. A polypeptide according to the invention is represented by an amino acid sequence. A nucleic acid construct according to the invention is defined as a polynucleotide which is isolated from a naturally occurring gene or which has been modified to contain segments of polynucleotides which are combined or juxtaposed in a manner which would not otherwise exist in nature.

The sequence information as provided herein should not be so narrowly construed as to require inclusion of erroneously identified bases. The skilled person is capable of identifying such erroneously identified bases and knows how to correct for such errors.

Expression is understood to include any (single) step involved in the production of a polypeptide including, but not limited to transcription, post-transcriptional modification, translation, post-translational modification, and secretion.

The term “expression construct” is interchangeably used herein with the terms “expression cassette” and is typically comprised of a polynucleotide according to the invention and the necessary components for expression of a polynucleotide such as a promoter, a terminator, a Kozak sequence etc. An expression construct may be located on a vector; such vector may be a plasmid.

A compound of interest in the context of all embodiments of the invention may be any biological compound. The biological compound may be biomass or a biopolymer or a metabolite. The biological compound may be encoded by a single polynucleotide or a series of polynucleotides composing a biosynthetic or metabolic pathway or may be the direct result of the product of a single polynucleotide or products of a series of polynucleotides, the polynucleotide may be a gene, the series of polynucleotide may be a gene cluster. In all embodiments of the invention, the single polynucleotide or series of polynucleotides encoding the biological compound of interest or the biosynthetic or metabolic pathway associated with the biological compound of interest, are preferred targets for the compositions and methods according to the invention. The biological compound may be native to the host cell or heterologous to the host cell.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid, polyamine, polyol, polypeptide (or polyamide), or polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. The term polypeptide refers to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. Polypeptides further include naturally occurring allelic and engineered variations of the above-mentioned polypeptides and hybrid polypeptides. The polypeptide may be native or may be heterologous to the host cell. The polypeptide may be a collagen or gelatine, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase. The polypeptide may also be an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, asparaginase, amylase, a maltogenic amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, galactolipase, chlorophyllase, polyphenoloxidase, ribonuclease, transglutaminase, or glucose oxidase, hexose oxidase, monooxygenase.

According to the invention, a compound of interest can be a polypeptide or enzyme with improved secretion features as described in WO2010/102982. According to the invention, a compound of interest can be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter(s) and terminator. The hybrid polypeptides may comprise a combination of partial or complete polypeptide sequences obtained from at least two different polypeptides wherein one or more may be heterologous to the host cell. Example of fusion polypeptides and signal sequence fusions are for example as described in WO2010/121933.

The biopolymer may be a polysaccharide. The polysaccharide may be any polysaccharide, including, but not limited to, a mucopolysaccharide (e. g., heparin and hyaluronic acid) and nitrogen-containing polysaccharide (e.g., chitin). In a preferred option, the polysaccharide is hyaluronic acid. A polynucleotide coding for the compound of interest or coding for a compound involved in the production of the compound of interest according to the invention may encode an enzyme involved in the synthesis of a primary or secondary metabolite, such as organic acids, carotenoids, (beta-lactam) antibiotics, and vitamins. Such metabolite may be considered as a biological compound according to the invention.

The term “metabolite” encompasses both primary and secondary metabolites; the metabolite may be any metabolite. Preferred metabolites are citric acid, gluconic acid, adipic acid, fumaric acid, itaconic acid and succinic acid.

A metabolite may be encoded by one or more genes, such as in a biosynthetic or metabolic pathway. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

A primary metabolite may be, but is not limited to, an amino acid, fatty acid, nucleoside, nucleotide, sugar, triglyceride, or vitamin.

A secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams. Other preferred metabolites are exo-metabolites. Examples of exo-metabolites are Aurasperone B, Funalenone, Kotanin, Nigragillin, Orlandin, Other naphtho-γ-pyrones, Pyranonigrin A, Tensidol B, Fumonisin B2 and Ochratoxin A.

The biological compound may also be the product of a selectable marker. A selectable marker is a product of a polynucleotide of interest which product provides for biocide or viral resistance, resistance to heavy metals, prototrophy to auxotrophs, and the like. Selectable markers include, but are not limited to, amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC (sulfate adenyltransferase), trpC (anthranilate synthase), ble (phleomycin resistance protein), hyg (hygromycin), NAT or NTC (Nourseothricin) as well as equivalents thereof.

According to the invention, a compound of interest is preferably a polypeptide as described in the list of compounds of interest.

According to another embodiment of the invention, a compound of interest is preferably a metabolite.

A cell according to the invention may already be capable of producing a compound of interest. A cell according to the invention may also be provided with a homologous or heterologous nucleic acid construct that encodes a polypeptide wherein the polypeptide may be the compound of interest or a polypeptide involved in the production of the compound of interest. The person skilled in the art knows how to modify a microbial host cell such that it is capable of producing a compound of interest.

All embodiments of the invention refer to a cell, not to a cell-free in vitro system; in other words, the systems according to the invention are cell systems, not cell-free in vitro systems.

In all embodiments of the invention, e.g., the cell according to the invention may be a haploid, diploid or polyploid cell.

A cell according to the invention is interchangeably herein referred as “a cell”, “a cell according to the invention”, “a host cell”, and as “a host cell according to the invention”; said cell may be any cell, e.g. a prokaryotic, an algae, a microalgae, marine eukaryote, a Labyrinthulomycetes or a eukaryotic cell. Preferably, the cell is not a mammalian cell.

When the cell is a prokaryotic cell, the prokaryotic host cell is preferably a bacterial host cell. The term “bacterial host cell” includes both Gram-negative and Gram-positive microorganisms. Preferably, a bacterial host cell according to invention is from a genus selected from the group consisting of Escherichia, Anabaena, Caulobactert, Gluconobacter, Rhodobacter, Pseudomonas, Paracoccus, Propionibacterium, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces. More preferably, the bacterial host cell is selected from the group consisting of B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Paracoccus denitrificans, Escherichia coli, Corynebacterium glutamicum, Staphylococcus carnosus, Streptomyces lividans, Sinorhizobium melioti and Rhizobium radiobacter.

Preferably the cell is a fungus, i.e. a yeast cell or a filamentous fungus cell. Preferably, the cell is deficient in an NHEJ (non-homologous end joining) component. Said component associated with NHEJ is preferably a homologue or orthologue of the yeast Ku70, Ku80, MRE11, RAD50, RAD51, RAD52, XRS2, SIR4, and/or LIG4. Alternatively, in the cell according to the invention NHEJ may be rendered deficient by use of a compound that inhibits RNA ligase IV, such as SCR7 (Vartak S V and Raghavan, FEBS J. 2015 November; 282(22):4289-94). The person skilled in the art knows how to modulate NHEJ and its effect on RNA-guided nuclease systems, see e.g. WO2014130955A1; Chu et al., Nat Biotechnol 2015, 33, 543-548; Yu et al., Cell Stem Cell, 2015, 16, 142-147.; all are herein incorporated by reference. The term “deficiency” is defined elsewhere herein.

When the cell according to the invention is a yeast cell, a preferred yeast cell is from a genus selected from the group consisting of Candida, Hansenula, Issatchenkia, Kluyveromyces, Pichia, Saccharomyces, Schizosaccharomyces, Yarrowia or Zygosaccharomyces; more preferably a yeast host cell is selected from the group consisting of Kluyveromyces lactis, Kluyveromyces lactis NRRL Y-1140, Kluyveromyces marxianus, Kluyveromyces. thermotolerans, Candida krusei, Candida sonorensis, Candida glabrata, Saccharomyces cerevisiae, Saccharomyces cerevisiae CEN.PK113-7D, Schizosaccharomyces pombe, Hansenula polymorpha, Issatchenkia orientalis, Yarrowia lipolytica, Yarrowia lipolytica CLIB122, Pichia stipidis and Pichia pastoris. A preferred yeast cell is Saccharomyces cerevisiae.

The host cell according to the invention is a filamentous fungal host cell. Filamentous fungi as defined herein include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK).

The filamentous fungal host cell may be a cell of any filamentous form of the taxon Trichocomaceae (as defined by Houbraken and Samson in Studies in Mycology 70: 1-51. 2011). In another preferred embodiment, the filamentous fungal host cell may be a cell of any filamentous form of any of the three families Aspergillaceae, Thermoascaceae and Trichocomaceae, which are accommodated in the taxon Trichocomaceae.

The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma. A preferred filamentous fungal host cell according to the invention is from a genus selected from the group consisting of Acremonium, Aspergillus, Chrysosporium, Myceliophthora, Penicillium, Talaromyces, Rasamsonia, Thielavia, Fusarium and Trichoderma; more preferably from a species selected from the group consisting of Aspergillus niger, Acremonium alabamense, Aspergillus awamori, Aspergillus foetidus, Aspergillus sojae, Aspergillus fumigatus, Talaromyces emersonii, Rasamsonia emersonii, Rasamsonia emersonii CBS393.64, Aspergillus oryzae, Chrysosporium lucknowense, Fusarium oxysporum, Mortierella alpina, Mortierella alpina ATCC 32222, Myceliophthora thermophila, Trichoderma reesei, Thielavia terrestris, Penicillium chrysogenum and P. chrysogenum Wisconsin 54-1255(ATCC28089); even more preferably the filamentous fungal host cell according to the invention is an Aspergillus niger.

When the host cell according to the invention is an Aspergillus niger host cell, the host cell preferably is CBS 513.88, CBS124.903 or a derivative thereof.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Preferred strains as host cells according to the present invention are Aspergillus niger CBS 513.88, CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255(ATCC28089), Penicillium citrinum ATCC 38065, Penicillium chrysogenum P2, Thielavia terrestris NRRL8126, Rasamsonia emersonii CBS393.64, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila C1, Garg 27K, VKM-F 3500 D, Chrysosporium lucknowense C1, Garg 27K, VKM-F 3500 D, ATCC44006 and derivatives thereof.

Preferably, a host cell according to the invention has a modification, preferably in its genome which results in a reduced or no production of an undesired compound as defined herein if compared to the parent host cell that has not been modified, when analysed under the same conditions.

A modification can be introduced by any means known to the person skilled in the art, such as but not limited to classical strain improvement, random mutagenesis followed by selection. Modification can also be introduced by site-directed mutagenesis.

Modification may be accomplished by the introduction (insertion), substitution (replacement) or removal (deletion) of one or more nucleotides in a polynucleotide sequence. A full or partial deletion of a polynucleotide coding for an undesired compound such as a polypeptide may be achieved. An undesired compound may be any undesired compound listed elsewhere herein; it may also be a protein and/or enzyme in a biological pathway of the synthesis of an undesired compound such as a metabolite. Alternatively, a polynucleotide coding for said undesired compound may be partially or fully replaced with a polynucleotide sequence which does not code for said undesired compound or that codes for a partially or fully inactive form of said undesired compound. In another alternative, one or more nucleotides can be inserted into the polynucleotide encoding said undesired compound resulting in the disruption of said polynucleotide and consequent partial or full inactivation of said undesired compound encoded by the disrupted polynucleotide.

In an embodiment the host cell according to the invention comprises a modification in its genome selected from
a) a full or partial deletion of a polynucleotide encoding an undesired compound,
b) a full or partial replacement of a polynucleotide encoding an undesired compound with a polynucleotide sequence which does not code for said undesired compound or that codes for a partially or fully inactive form of said undesired compound.
c) a disruption of a polynucleotide encoding an undesired compound by the insertion of one or more nucleotides in the polynucleotide sequence and consequent partial or full inactivation of said undesired compound by the disrupted polynucleotide.

This modification may for example be in a coding sequence or a regulatory element required for the transcription or translation of said undesired compound. For example, nucleotides may be inserted or removed so as to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32(7) or Gupta et al. (1968), Proc. Natl. Acad. Sci USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of site-directed mutagenesis procedures are the QuickChange™ site-directed mutagenesis kit (Stratagene Cloning Systems, La Jolla, Calif.), the ‘The Altered Sites® II in vitro Mutagenesis Systems’ (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995 Horizon Scientific Press, PO Box 1, Wymondham, Norfolk, U.K.).

Preferred methods of modification are based on recombinant genetic manipulation techniques such as partial or complete gene replacement or partial or complete gene deletion.

For example, in case of replacement of a polynucleotide, nucleic acid construct or expression cassette, an appropriate DNA sequence may be introduced at the target locus to be replaced. The appropriate DNA sequence is preferably present on a cloning vector. Preferred integrative cloning vectors comprise a DNA fragment, which is homologous to the polynucleotide and/or has homology to the polynucleotides flanking the locus to be replaced for targeting the integration of the cloning vector to this pre-determined locus. In order to promote targeted integration, the cloning vector is preferably linearized prior to transformation of the cell. Preferably, linearization is performed such that at least one but preferably either end of the cloning vector is flanked by sequences homologous to the DNA sequence (or flanking sequences) to be replaced. This process is called homologous recombination and this technique may also be used in order to achieve (partial) gene deletion.

For example a polynucleotide corresponding to the endogenous polynucleotide may be replaced by a defective polynucleotide; that is a polynucleotide that fails to produce a (fully functional) polypeptide. By homologous recombination, the defective polynucleotide replaces the endogenous polynucleotide. It may be desirable that the defective polynucleotide also encodes a marker, which may be used for selection of transformants in which the nucleic acid sequence has been modified. Alternatively or in combination with other mentioned techniques, a technique based on recombination of cosmids in an E. coli cell can be used, as described in: A rapid method for efficient gene replacement in the filamentous fungus Aspergillus nidulans (2000) Chaveroche, M-K, Ghico, J-M. and d'Enfert C; Nucleic acids Research, vol 28, no 22.

Alternatively, modification, wherein said host cell produces less of or no protein such as the polypeptide having amylase activity, preferably α-amylase activity as described herein and encoded by a polynucleotide as described herein, may be performed by established anti-sense techniques using a nucleotide sequence complementary to the nucleic acid sequence of the polynucleotide. More specifically, expression of the polynucleotide by a host cell may be reduced or eliminated by introducing a nucleotide sequence complementary to the nucleic acid sequence of the polynucleotide, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl. Environ. Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52.).

A modification resulting in reduced or no production of undesired compound is preferably due to a reduced production of the mRNA encoding said undesired compound if compared with a parent microbial host cell which has not been modified and when measured under the same conditions. A modification which results in a reduced amount of the mRNA transcribed from the polynucleotide encoding the undesired compound may be obtained via the RNA interference (RNAi) technique (Mouyna et al., 2004). In this method identical sense and antisense parts of the nucleotide sequence, which expression is to be affected, are cloned behind each other with a nucleotide spacer in between, and inserted into an expression vector. After such a molecule is transcribed, formation of small nucleotide fragments will lead to a targeted degradation of the mRNA, which is to be affected. The elimination of the specific mRNA can be to various extents. The RNA interference techniques described in e.g. WO2008/053019, WO2005/05672A1 and WO2005/026356A1.

A modification which results in decreased or no production of an undesired compound can be obtained by different methods, for example by an antibody directed against such undesired compound or a chemical inhibitor or a protein inhibitor or a physical inhibitor (Tour O. et al, (2003) Nat. Biotech: Genetically targeted chromophore-assisted light inactivation. Vol. 21. no. 12:1505-1508) or peptide inhibitor or an anti-sense molecule or RNAi molecule (R. S. Kamath et al, (2003) Nature: Systematic functional analysis of the Caenorhabditis elegans genome using RNAi. Vol. 421, 231-237).

In addition of the above-mentioned techniques or as an alternative, it is also possible to inhibiting the activity of an undesired compound, or to re-localize the undesired compound such as a protein by means of alternative signal sequences (Ramon de Lucas, J., Martinez O, Perez P., Isabel Lopez, M., Valenciano, S. and Laborda, F. The Aspergillus nidulans carnitine carrier encoded by the acuH gene is exclusively located in the mitochondria. FEMS Microbiol Lett. 2001 Jul. 24; 201(2):193-8.) or retention signals (Derkx, P. M. and Madrid, S. M. The foldase CYPB is a component of the secretory pathway of Aspergillus niger and contains the endoplasmic reticulum retention signal HEEL. Mol. Genet. Genomics. 2001 December; 266(4):537-545), or by targeting an undesired compound such as a polypeptide to a peroxisome which is capable of fusing with a membrane-structure of the cell involved in the secretory pathway of the cell, leading to secretion outside the cell of the polypeptide (e.g. as described in WO2006/040340).

Alternatively, or in combination with above-mentioned techniques, decreased or no production of an undesired compound can also be obtained, e.g. by UV or chemical mutagenesis (Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol Gen Genet. 1992 August; 234(2):332-6.) or by the use of inhibitors inhibiting enzymatic activity of an undesired polypeptide as described herein (e.g. nojirimycin, which function as inhibitor for β-glucosidases (Carrel F. L. Y. and Canevascini G. Canadian Journal of Microbiology (1991) 37(6): 459-464; Reese E. T., Parrish F. W. and Ettlinger M. Carbohydrate Research (1971) 381-388)). In an embodiment of the invention, the modification in the genome of the host cell according to the invention is a modification in at least one position of a polynucleotide encoding an undesired compound.

A deficiency of a cell in the production of a compound, for example of an undesired compound such as an undesired polypeptide and/or enzyme is herein defined as a mutant microbial host cell which has been modified, preferably in its genome, to result in a phenotypic feature wherein the cell: a) produces less of the undesired compound or produces substantially none of the undesired compound and/or b) produces the undesired compound having a decreased activity or decreased specific activity or the undesired compound having no activity or no specific activity and combinations of one or more of these possibilities as compared to the parent host cell that has not been modified, when analysed under the same conditions.

Preferably, a modified host cell according to the invention produces 1% less of the un-desired compound if compared with the parent host cell which has not been modified and measured under the same conditions, at least 5% less of the un-desired compound, at least 10% less of the un-desired compound, at least 20% less of the un-desired compound, at least 30% less of the un-desired compound, at least 40% less of the un-desired compound, at least 50% less of the un-desired compound, at least 60% less of the un-desired compound, at least 70% less of the un-desired compound, at least 80% less of the un-desired compound, at least 90% less of the un-desired compound, at least 91% less of the un-desired compound, at least 92% less of the un-desired compound, at least 93% less of the un-desired compound, at least 94% less of the un-desired compound, at least 95% less of the un-desired compound, at least 96% less of the un-desired compound, at least 97% less of the un-desired compound, at least 98% less of the un-desired compound, at least 99% less of the un-desired compound, at least 99.9% less of the un-desired compound, or most preferably 100% less of the un-desired compound.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The invention is further illustrated by the following examples.

### EXAMPLES

In the following Examples, various embodiments of the invention are illustrated. From the above description and these Examples, one skilled in the art can make various changes and modifications of the invention to adapt it to various usages and conditions.

### Material and Methods

General Molecular Biology Techniques

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

Plasmids, Oligonucleotide Primers and Strains

Plasmids used in the examples are listed in Table 1. Strains used for further strain engineering are listed in Table 2.

Media

Media used in the experiments were YEPh-medium (10 g/l yeast extract, 20 g/l phytone peptone (BD BioSciences, Temse, Belgium) and solid YNB-medium (6.7 g/l yeast nitrogen base, 15 g/l agar), supplemented with sugars (i.e., YEPhD, 20 g/l glucose; YEPhG, 20 g/l galactose). For solid YEPh medium, 15 g/l agar was added to the liquid medium prior to sterilization.

For the CuSO₄ induction experiments, mineral medium was used. The composition of mineral medium has been described by Verduyn et al., (Yeast, 1992, volume 8, pp. 501-517). Ammonium sulphate was replaced by 2.3 g/l urea as a nitrogen source. Initial pH of the medium was 4.6.

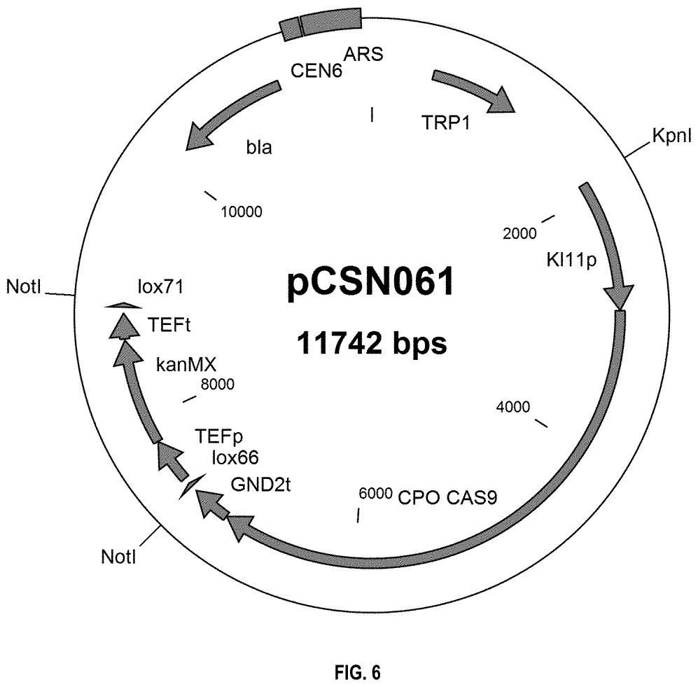

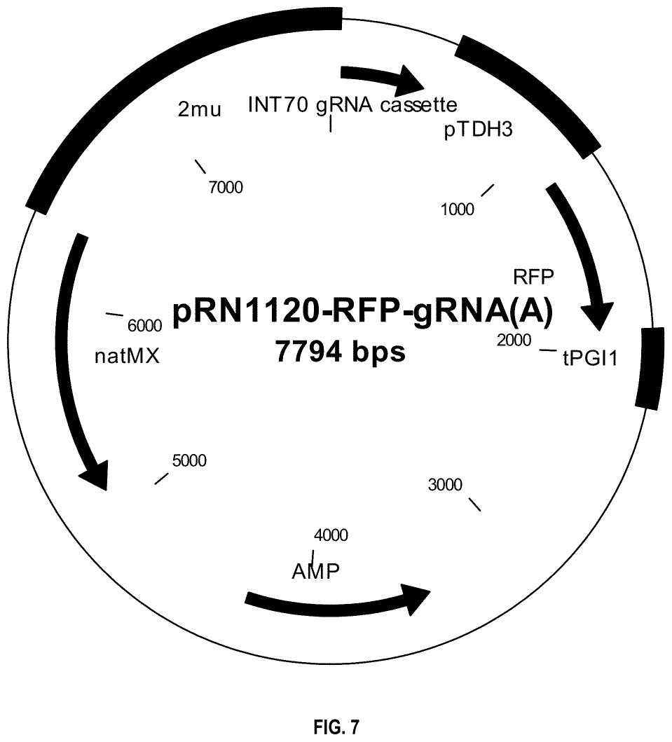

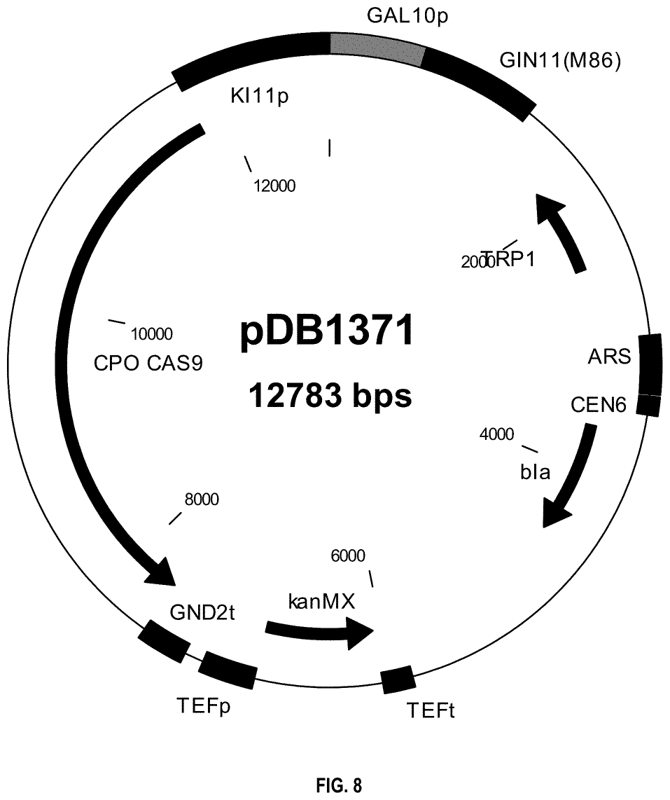

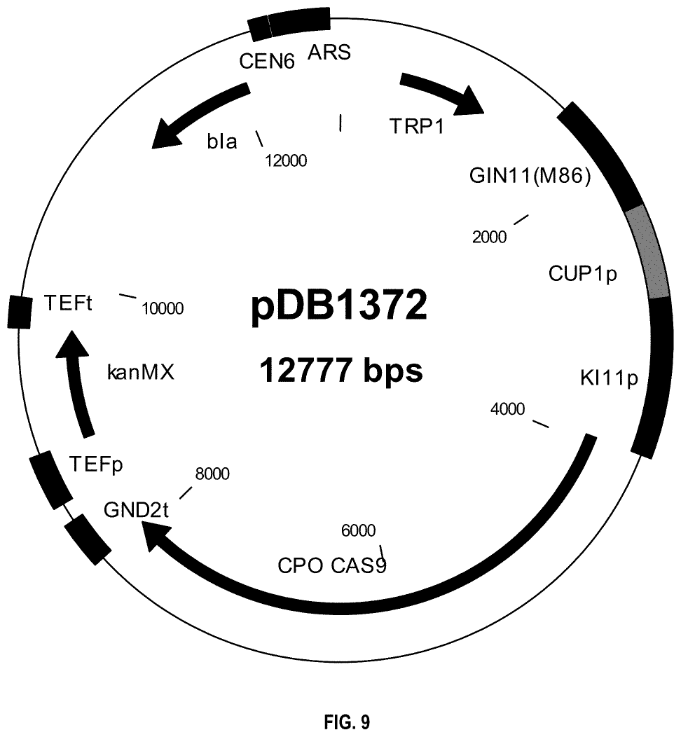

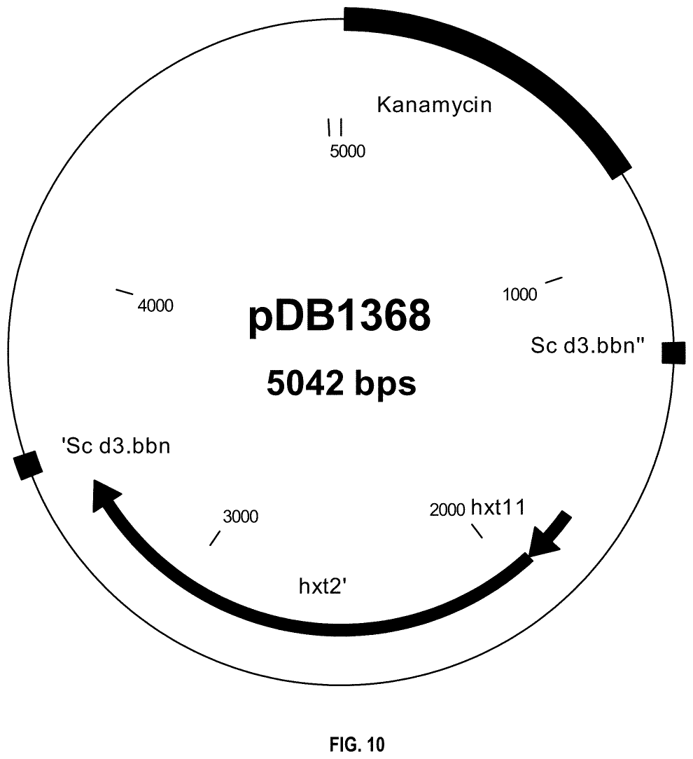

TABLE 1
Listing of plasmids used in examples.

| Name | Characteristics | Origin |
|---|---|---|
| pCSN061 | CEN6.ARSH4, kanMX, Cas9. | PCT/EP2016/ |
| | | 050136 |
| | | SEQ ID NO: 18, |
| | | FIG. 6. |
| pRN1120- | natMX-bearing shuttle | SEQ ID NO: 1, |
| RFP- | vector based on pRS305 with 2- | FIG. 7 |
| gRNA(A) | micron origin and expression | |
| | cassette TDH3p-RFP-PGI1t. | |
| pDB1371 | pCSN061 with pGAL10-GIN11(M86) | Example 1 |
| | inserted in the KpnI/NgoMIV site. | SEQ ID NO: 4, |
| | | FIG. 8 |
| pDB1372 | pCSN061 with pCUP1-GIN11(M86) | Example 1 |
| | inserted in the KpnI/NgoMIV site. | SEQ ID NO: 5, |
| | | FIG. 9 |
| pDB1368 | Cloning vector with expression | Example 2 |
| | cassette | SEQ ID NO: 6, |
| | SparTDH3p-HXT11/2^{N366T}-EFM1t. | FIG. 10 |

TABLE 2
Listing of S. cerevisiae strains used and generated in the examples.

| Strain name | Relevant Genotype | Origin |
|---|---|---|
| CEN.PK113-7D | MATa URA3 HIS3 LEU2 TRP1 MAL2-8 SUC2 | Van Dijken et al., Enzyme |
| | | Microb Technol. 2000 Jun. |
| | | 1; 26(9-10): 706-714. |
| CP-61 | CEN.PK113-7D pCSN061 | Example 2 |
| | (pool of 6 transformants) | |
| CP-71 | CEN.PK113-7D pDB1371 | Example 2 |
| | (pool of 6 transformants) | |
| CP-72 | CEN.PK113-7D pDB1372 | Example 2 |
| | (pool of 6 transformants) | |
| CP-61-HXT | CEN.PK113-7D int70::[SparTDH3p-HXT11/2^{N366T}- | Example 2, 4 |
| | EFM1t] pCSN061 | |
| CP-71-HXT | CEN.PK113-7D int70::[SparTDH3p-HXT11/2^{N366T}- | Example 2, 3, 4 |
| | EFM1t] pDB1371 | |
| CP-72-HXT | CEN.PK113-7D int70::[SparTDH3p-HXT11/2^{N366T}- | Example 2, 5 |
| | EFM1t] pDB1372 | |

### Example 1: Cloning GIN11M86-Bearing Cas9 Expression Plasmids

To be able to control the expression of GIN11(M86), the sequence was designed upstream of well-known inducible promoters in Saccharomyces cerevisiae: the 600 bp upstream sequences of GAL10 (Partow et al., Yeast. 2010 November; 27(11):955-964) or CUP1 (Mascorro-Gallardo et al., Gene. 1996 Jun. 12; 172(1):169-170). GAL10 being suppressed by glucose and induced by galactose, and the CUP1 promoter induced by copper. Both pGAL10-GIN11(M86) (cFS0017; SEQ ID NO: 2) and pCUP1-GIN11(M86) (cFS0018; SEQ ID NO: 3) expression cassettes were synthesized at ATUM (Menlo Park, Calif., USA). KpnI and NgoMIV sites were added to the pGAL10-GIN11(M86) construct by extension PCR amplification using a forward primer (SEQ ID NO: 7) and a reverse primer (SEQ ID NO: 8) after which the PCR product was cloned into pCSN061, the plasmid bearing a S. pyogenes Cas9 expression cassette, using the abovementioned restriction sites, resulting in plasmid pDB1371 (SEQ ID NO: 4). The pCUP1-GIN11(M86) cassette was cloned in pCSN061 using KpnI and NgoMIV sites which were part of the synthesized construct, resulting in plasmid pDB1372 (SEQ ID NO: 5). The pGAL10 promoter sequence used is set out in SEQ ID NO: 19, the pCUP1 promoter sequence used is set out in SEQ ID NO: 20. The GIN11(M86) sequence is set out in SEQ ID NO: 21.

### Example 2: GIN11M86-Bearing Cas9 Expression Plasmids Facilitate Genome Modifications Efficiently

The followed strain construction approach is described in patent application PCT/EP2013/056623 and PCT/EP2016/050136. PCT/EP2013/056623 describes the techniques enabling the construction of expression cassettes from various genes of interest in such a way, that these cassettes are combined into a pathway and integrated in a specific locus of the yeast genome upon transformation of this yeast.

Example 9 of PCT/EP2016/050136 describes the use of a CRISPR-Cas9 system for integration of expression cassettes into the genome of a host cell, in this case S. cerevisiae. In a first transformation round, pCSN061, pDB1371 or pDB1372, each being a G418-selectable episomal plasmid bearing the S. pyogenes Cas9 expression cassette, were individually introduced to yeast. CEN.PK113-7D was transformed with 500 ng of either pCSN061, pDB1371 or pDB1372. Correct transformants were selected on solid agar YEPhD medium supplemented with 200 micrograms per milliliter G418 (YEPhD-G418, Invivogen). Subsequently, several transformants were re-streaked on YEPhD-G418 (200 micrograms per milliliter) agar to obtain pure colonies. Six colonies were pooled to continue to the next transformation round. The three resulting transformant pools were named: CP-61, CP-71, and CP-72, respectively (see Table 2).

In a second transformation round, to cells pre-expression Cas9, a gRNA-recipient plasmid backbone PCR fragment, a guide RNA PCR fragment with homology to the gRNA-recipient plasmid backbone PCR fragment which allows in vivo recombination into a circular plasmid containing a nourseothricin selection marker, and a donor DNA expression cassette were transformed, resulting in the intended modifications. To introduce the donor DNA expression cassette containing the intended modifications to the yeast genome, an integration site in the yeast genome was selected. DNA flanks with approximately 50 bp homology to the selected integration site were added to the donor DNA by extension PCR using primers introducing flanking sequences to the generated PCR products. These flanks (50 bp in size at the 5′ and 3′ end of the donor DNA expression cassette) allow for correct integration of the donor DNA fragment to the intended locus upon transformation in yeast. Upon transformation of yeast cells with the DNA fragments, in vivo recombination and integration into the genome takes place at the desired location.

Integration site: the expression cassette was targeted at the INT70 locus. The INT70 integration site is a non-coding region between YNL180C and YNL178W located on chromosome XIV of S. cerevisiae. The guide sequence to target INT70 was designed with a gRNA designer tool (https://www.dna20.com/eCommerce/cas9/input).

The gRNA expression cassette (as described by DiCarlo et al., Nucleic Acids Res. 2013 April; 41(7):4336-4343) was ordered as synthetic DNA cassette (gBLOCK) at Integrated DNA Technologies (Leuven, Belgium. INT70 gBLOCK; SEQ ID NO: 9).

gRNA-recipient plasmid backbone: In vivo assembly of the gRNA expression plasmid is subsequently completed by co-transforming a linear PCR fragment derived from yeast vector pRN1120-RFP-gRNA(A). pRN1120-RFP-gRNA(A) is a multi-copy yeast shuttling vector that contains a functional natMX marker cassette conferring resistance against nourseotricin (NTC) (SEQ ID NO: 1, FIG. 7). The backbone of this plasmid is based on pRS305 (Sikorski and Hieter, Genetics 1989, vol. 122, pp. 19-27), including a functional 2-micron ORI sequence, functional natMX marker cassette, and a RFP expression cassette to be able to track colonies that harbor the plasmid based on fluorescence or by pink to purple coloration of the colonies visible by eye.

Donor DNA expression cassette construction: the open reading frames (ORFs), promoter sequences and terminators were synthesized at ATUM (Menlo Park, Calif., USA). The promoter, ORF and terminator sequences were recombined by using the Golden Gate technology, as described by Engler et al., PLoS One. 2008; 3(11): e3647 and Engler et al., PLoS One. 2009; 4(5): e5553 and references therein. The expression cassettes were cloned into a standard cloning vector. The resulting plasmid (also listed in Table 1) is pDB1368. pDB1368 (SEQ ID NO: 6) bears an expression cassette for the chimeric pentose transporter HXT11/2 (Shin et al., 2017, Biotechnol Bioeng. 2017, September; 114(9):1937-1945. doi: 10.1002/bit.26322) under control of the Saccharomyces paradoxus TDH3 promoter (SparTDH3p) and Saccharomyces cerevisiae EFM1 terminator (EFM1t). Flanks for integration into the INT70 locus were added to the 5′ and 3′ end of the donor DNA expression cassette by extension PCR as described below.

Transformation of CP-61, CP-71, CP-72 with Specified PCR Fragments

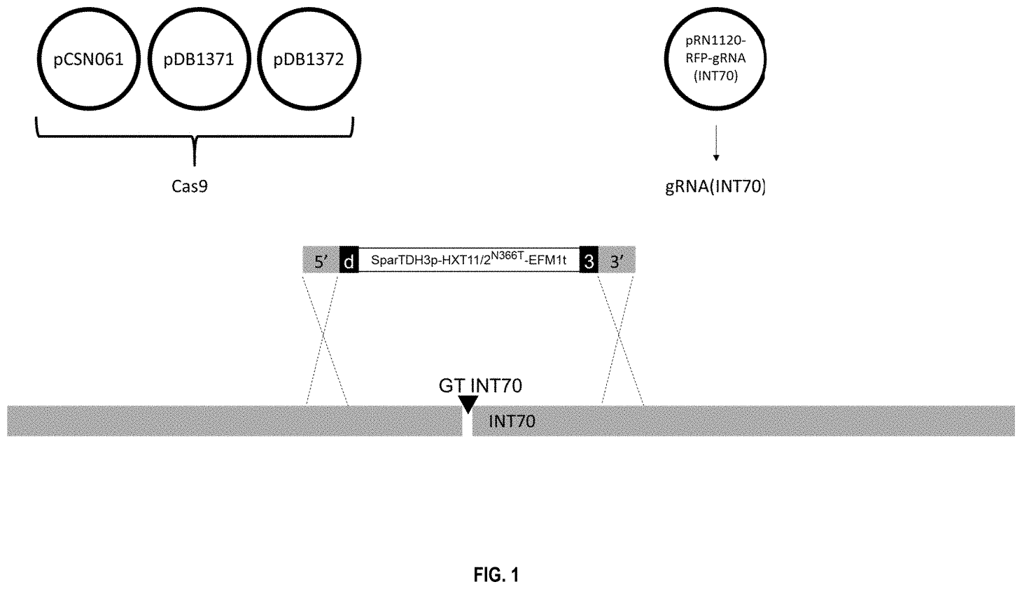

For the second transformation round, CP-61, CP-71 and CP-72, pre-expressing Cas9, were transformed with the following fragments resulting in the assembly of the HXT11/2^{N366T} expression cassette and integration at the INT70 locus (FIG. 1):
1) A donor DNA expression cassette PCR fragment (int70[5′]-conD-HXT11/2-con3-int70[3′]) generated with a forward primer (SEQ ID NO: 10) and a reverse primer (SEQ ID NO: 11) using pDB1368 as template;
2) A gRNA-recipient plasmid backbone PCR fragment (backbone-1120-RFP-gRNA[A]) generated with a forward primer (SEQ ID NO: 12) and a reverse primer (SEQ ID NO: 13) using pRN1120-RFP-gRNA(A) (SEQ ID NO: 1) as template;
3) A guide RNA PCR fragment (gRNA-INT70) generated with a forward primer (SEQ ID NO: 14) and a reverse primer (SEQ ID NO: 15) using INT70 gBLOCK (SEQ ID NO: 9) as template.

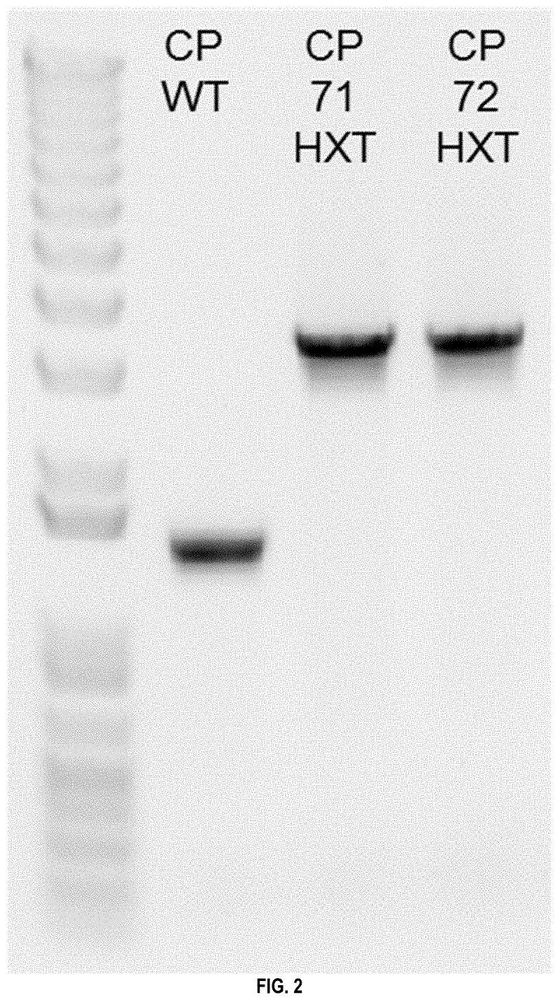

Transformants were selected on YEPhD-agar plates containing 200 micrograms/ml G418 and 200 micrograms/ml NTC (Werner Bioagents) (YEPhD-G418-NTC). Diagnostic PCR with forward primer (SEQ ID NO: 16) and reverse primer (SEQ ID NO: 17) was performed on genomic DNA isolated from one colony per transformation to confirm the correct assembly and integration of the HXT11/2 expression cassettes at the INT70 locus (FIG. 2). These results indicated that the Cas9-plasmids, pDB1371 and pDB1372 were functional and enabled the correct, CRISPR/Cas9-mediated integration of the HXT11/2 expression cassette at the INT70 locus. Resulting colonies for further plasmid loss experiments for pCSN061, pDB1371 or pDB1372, were named CP-61-HXT, CP-71-HXT, or CP-72-HXT, respectively (Table 2).

### Example 3: Galactose-Induced Plasmid Loss from CP-71-HXT Indicated More Efficient Plasmid Loss when Inducing GIN11(M86) Sequence

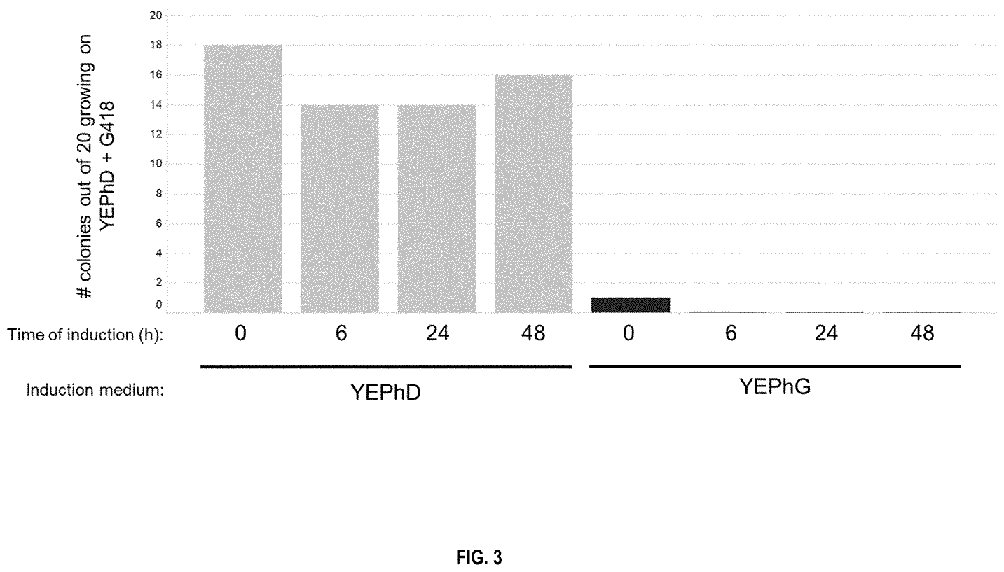

To compare the efficiency of plasmid loss by inducing the GAL10p-GIN11(M86) construct present on pDB1371 with and without induction, CP-71-HXT colonies from YEPhD-G418-NTC agar plates were induced on liquid YEPhG medium for 0, 6, 24 and 48 hours, 30° C., 250 rpm in shake flask. For the 0 hours condition, cells were plated directly on YEPhG agar medium. As control condition, colonies were also cultivated in liquid YEPhD medium in which no induction of GAL10p-GIN11(M86) should take place. Based on the OD600, the cultures were diluted to obtain single colonies on plate by plating on YEPhG or YEPhD agar medium depending on previous induction medium. After 2 days of growth at 30° C., 20 colonies per condition were re-plated to YEPhD-G418 and YEPhD to score the efficiency of plasmid loss (FIG. 3). Colonies unable to grow on YEPhD-G418, but able to grow on YEPhD agar plates have lost the Cas9-expression plasmid (pDB1371). Without induction (YEPhD medium), ˜75% of the colonies retains the Cas9 plasmid. When induced on YEPhG, all colonies lost the Cas9 plasmid. Even without induction and growth on liquid YEPhG, timepoint 0 hrs, only 1 out of 20 colonies grows on G418. This indicates that plating on YEPhG plates should be sufficient to select for colonies that have lost the Cas9 plasmid pDB1371.

### Example 4: Direct Induction of GIN11(M86) by Plating Cells on YEPhG

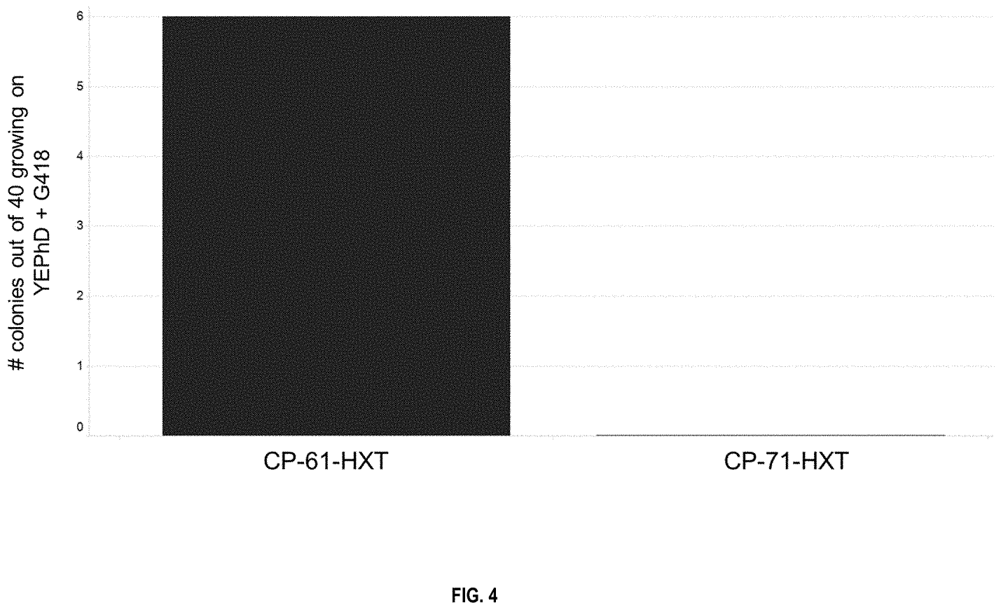

With the results of the first experiment (Example 3) in mind, a second experiment was performed in which colonies were transferred to YEPhG plates without previous induction in liquid YEPhG medium (FIG. 4). To determine the specific effect of the GIN11(M86) sequence, CP-61-HXT cells which contain pCSN061, a Cas9 expression plasmid that does not harbor the GIN11(M86) sequence, were taken along as a control. Forty transformants from the YEPhD-G418-NTC transformation plates were directly streaked onto YEPhG plates. Subsequently, after 2 days of growth at 30° C., the 40 colonies were streaked onto YEPhD and YEPhD-G418 agar plates and grown again for 2 days at 30° C. 6 out of 40 colonies transformed with pCSN061 (CP-61-HXT) were still able to grow on YEPhD-G418 agar, indicating that the Cas9-containing plasmid was still present some of the CP-61-HXT colonies. No colonies transformed with pDB1371 (CP-71-HXT) appeared on YEPhD-G418 agar plates, indicating all CP-71-HXT colonies lost the Cas9 GIN11(M86) containing plasmid. 40 re-streaks of CP-61-HXT and 40 re-streaks of CP-71-HXT grew on YEPhD plates as expected. Since there is no induction in liquid medium required, the pDB1371 plasmid can be removed from CEN.PK113-7D within only 2 days growth on a YEPhG agar plate.

### Example 5: Copper-Induced Plasmid Loss of CP-61-HXT and CP-72-HXT

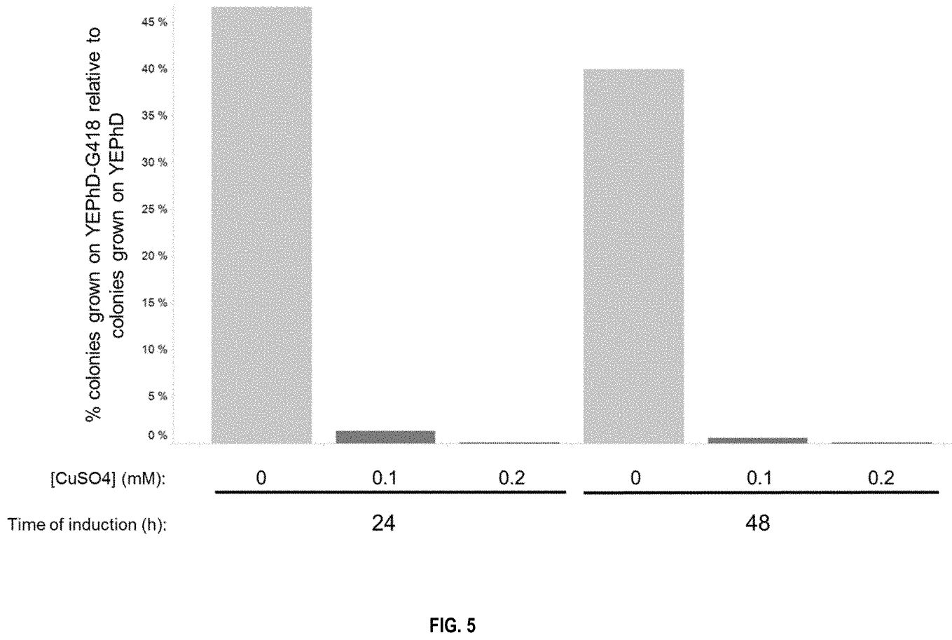

To compare the efficiency of plasmid loss by inducing the CUP1p-GIN11(M86) construct present on pDB1372 with and without induction, CP-72-HXT colonies from YEPhD-G418-NTC agar plates were not induced (0 mM CuSO₄) or induced on mineral medium supplemented with 20 g/l glucose, and 0.1 or 0.2 mM CuSO₄ for 24 and 48 hours. Colonies were subsequently plated on YEPhD-G418 and YEPhD agar medium. After 2 days of growth at 30° C., the number of colonies were counted per condition to score the relative efficiency of plasmid loss compared to the no-induction condition (mineral medium with 20 g/l glucose and no CuSO₄) plated on YEPhD. For CP-62-HXT induced on 0.1 mM or 0.2 mM CuSO₄, >90% of the cells has lost the Cas9 plasmid after 24 h of induction (FIG. 5). After 48 h of induction, the percentages are comparable, but maybe slightly more cells lost the pDB1372 plasmid after longer induction (FIG. 5). The results indicate that the Cas9-containing plasmid pDB1372 is efficiently lost upon induction by CuSO₄ from CP-72-HXT.

### IN SUMMARY

The classical way of removing plasmids containing dominant antibiotic resistance markers, like KanMX, in prototrophic strains is based on spontaneous loss during cell division. This classical procedure takes 6 and has varying success rates (0-100%). To increase control over this process the GIN11(M86) suicide gene was added to the Cas9-containing plasmid, which enables active selection against the plasmid. The examples describe the induction of the GIN11(M86) sequence via two inducible systems:

If GIN11(M86) is coupled to the GAL10 promoter, the Cas9 plasmid can be removed with an efficiency of 100% in 2 days when plating directly on YEPhG to induce expression of GIN11(M86) (Example 4).

If GIN11(M86) is coupled to the CUP1 promoter, the Cas9 plasmid can be removed with an efficiency of (Example 5). Induction in 0.1 mM CuSO₄ for 24 hours works most efficient.

1. A method for genome editing in a host cell comprising:
a) contacting a host cell with:
i) an expression construct comprising a polynucleotide that has a negative influence on the viability of the host cell when expressed, operably linked to an inducible promoter,
ii) a functional heterologous genome editing enzyme, or an expression construct capable of expressing a functional heterologous genome editing enzyme in the host cell,
(iii) a guide-polynucleotide, or an expression construct capable of expressing a guide-polynucleotide in the host cell, and, optionally,
(iv) an exogenous polynucleotide,
b) culturing the host cell under conditions that induce genome editing, and
c) culturing the host cell under conditions that induce the expression of the polynucleotide that has a negative influence on the viability of the host cell;
wherein at least an expression construct capable of expressing the functional heterologous genome editing enzyme in the host cell or an expression construct capable of expressing the guide-polynucleotide in the host cell is located on the expression construct comprising the polynucleotide that has a negative influence on the viability of the host cell when expressed.

2. The method according to claim 1, wherein the host cell is a prokaryotic host cell, a eukaryotic host cell, a marine eukaryote, a microalgae or an algae host cell.

3. The method according to claim 2, wherein the host cell is a eukaryotic host cell and optionally is a fungal host, optionally a yeast or a filamentous fungal host cell.

4. The method according to claim 3, wherein the yeast cell is a Saccharomyces host cell, optionally a Saccharomyces cerevisiae host cell.

5. The method according to claim 1, wherein the expression construct comprising the polynucleotide that has a negative influence on the viability of the host cell when expressed, is present on an episomal entity, optionally a plasmid.

6. The method according to claim 1, wherein the genome editing enzyme is a Cas-like enzyme.

7. The method according to claim 1, wherein the inducible promoter is a copper inducible promoter, optionally a CUP1 promoter or a galactose inducible promoter, optionally a GAL10 promoter.

8. The method according to claim 8, wherein the CUP1 promoter has at least 80% sequence identity with SEQ ID NO: 20 and/or wherein the GAL10 promoter has at least 80% sequence identity with SEQ ID NO: 19

9. The method according to claim 1, wherein the polynucleotide that has a negative influence on the viability of the host cell when expressed has at least 80% sequence identity with SEQ ID NO: 21.

10. A host cell obtainable by or obtained by the method according to claim 1.

11. A method for production of a compound of interest comprising culturing a host cell according to claim 10 under conditions conducive to expression of the compound of interest and, optionally, isolating and/or purifying the compound of interest.

12. A method for production of a compound of interest comprising performing the method according to claim 1 and subsequently culturing said host cell under conditions conducive to expression of the compound of interest and, optionally, isolating and/or purifying the compound of interest.
